(19) United States
(12) Patent Application Publication
(10) Pub. No.: US 2023/0325944 A1
(43) Pub. Date: Oct. 12, 2023

(54) ADAPTIVE WELLNESS COLLABORATIVE MEDIA SYSTEM

(71) Applicant: PEOPLE FIRST TECHNOLOGIES, INC., Simsbury, CT (US)

(72) Inventors: Cécile DEJOUX, Paris (FR); Pierre DEJOUX, Paris (FR)

(21) Appl. No.: 18/131,398

(22) Filed: Apr. 6, 2023

Related U.S. Application Data
(60) Provisional application No. 63/327,972, filed on Apr. 6, 2022.

(51) Int. Cl. G06Q 50/00; G06N 3/08

(52) U.S. Cl. CPC G06Q 50/01; G06N 3/08

(57) ABSTRACT

Systems, methods, or devices may generate personalized wellness recommendations which may be in association with group characteristic or user characteristic. Recommendations may include energizer recommendations, which may be generated by machine learning algorithms.

### CROSS-REFERENCE TO RELATED APPLICATIONS

This application claims the benefit of U.S. Provisional Patent Application No. 63/327,972, filed Apr. 6, 2022, entitled “Adaptive Wellness System,” the entire content of which is incorporated herein by reference.

### TECHNOLOGICAL FIELD

The present disclosure generally relates to methods, apparatuses, and computer program products for a collaborative platform.

### BACKGROUND

Flexible work models that combine remote and in-office work (e.g., hybrid work environments) may be considered disjointed, causing geographically distant workers to have limited visibility to moments outside meetings where people or coworkers may connect. Flexible work arrangements may lack chance beneficial occurrences between people or coworkers that may add some familiarity with the workplace and foster work relationships. The lack of these chance occurrences or serendipity may lead to feelings of isolation and loneliness of workers, which in turn may negatively impact knowledge workers' sense of belonging, personal productivity, team success, and job satisfaction. Overall, such arrangements may affect a worker's wellbeing or sense of wellness.

### BRIEF SUMMARY

Various systems, methods, or devices are described for generating personalized wellness recommendations which may be associated with group characteristic or user characteristic. Recommendations may include energizer recommendations, which may be generated by machine learning.

In various examples, systems or methods may receive an indication of a user's input associated with the user, such as mood, energy level, level of engagement, work environment, energizer profile, and the like. A list of group characteristics may determine when an energizer may be needed with the received inputs. An energizer may be referred to as an activity (e.g., yoga) that may energize a user associated with a user profile. A machine learning module may develop a set of effective energizers associated with the list of group characteristics and the energizer profile. The machine learning module may utilize a neural network to develop an association between an energizer profile, a list of group characteristics, and a set of effective energizers. An energizer recommendation may be generated based on an association between the list of group characteristics, the energizer profile, and effective energizers. A machine learning module, which may be the same or a different machine learning module may generate the energizer recommendation. The effective organizers may include energizers associated or with preferred energizers that may have elevated user mood, energy level, engagement level, overall wellness, or the like. The energizer recommendations may be provided via the adaptive wellness platform, for example, on a graphical user interface, communication device, or computing device assessing the adaptive wellness platform.

In various examples, the list of group characteristics module may identify one or more behavioral category associated with 5R that affects the weight of a user's mood, energy level, level of engagement, or any combination thereof to determine the recommended energizer. The reception of information, associated with an energizer profile, operations may include one or more modules to analyze inputs (e.g., mood, energy level, preferred energizers, level of engagement, feedback information or any combination thereof) within a time range, or search for particular inputs above a predetermined threshold. The effective energizer may relate to one or more input increasing or improving its relative level in response to an energizer. Inputs (e.g., wellness information) from which the energizer profile is created may include one or more mood, energy level, preferred energizers, level of engagement, feedback information, location associated with the user, or the like. Inputs may be received from a memory of a user device, data store of the adaptive wellness system, a profile on the adaptive wellness system, or the like, for example, and within a time range. The time range may be within a past number of hours, days, months, or years. The time range may be selected based on a time range associated with or defined by a group or the adaptive wellness platform.

The machine learning module may be trained based on a set of energizers associated with one or more group characteristics or energizer profiles. In various examples, the machine learning module may utilize one or more neural network to develop associations between energizer profiles, feedback information, or a set of effective energizers. An energizer recommendation to users associated with a company (e.g., group) may be generated and provided on a graphical user interface of a device (e.g., computing device, communication device, or the like). The product recommendation may be in the form of an image, video, text, email, message, or any combination thereof.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

The summary, as well as the following detailed description, is further understood when read in conjunction with the appended drawings. For the purpose of illustrating the disclosed subject matter, there are shown in the drawings examples of the disclosed subject matter; however, the disclosed subject matter is not limited to the specific methods, compositions, and devices disclosed. In addition, the drawings are not necessarily drawn to scale. In the drawings:

FIG. 1 illustrates an example system that may implement an adaptive wellness platform.

FIG. 2 illustrates an example device, in accordance with an example of the present disclosure.

FIG. 3 illustrates an example user interface in accordance with an example of the present disclosure.

FIG. 4 illustrates vectors utilized to create a value associated with mood, in accordance with an example of the present disclosure.

FIGS. 5A, 5B, and 5C illustrate an example mood capture process associated with a user, in accordance with an example of the present disclosure.

FIGS. 6A, 6B, and 6C illustrate an example initial energy level capture process associated with a user, in accordance with an example of the present disclosure.

FIG. 7 illustrates an example alert process associated with mood and energy level of a user, in accordance with an example of the present disclosure.

FIG. 8 illustrates an alternate example alert process associated with mood, energy level, and engagement level of a user, in accordance with an example of the present disclosure.

FIG. 9 illustrates an example method of initial energizer recommendation, in accordance with an example of the present disclosure.

FIG. 10 illustrates an example method of recommending energizers associated with a user, in accordance with an example of the present disclosure.

FIG. 11 illustrates a flow chart for monitoring wellness associated with a user, in accordance with an example of the present disclosure.

FIG. 12 illustrates a flow chart for generating an energizer recommendation, in accordance with an example of the present disclosure.

FIG. 13 illustrates a machine learning and training model, in accordance with an example of the present disclosure.

FIG. 14 illustrates an individual level method associated with a function, in accordance with an example of the present disclosure.

FIG. 15 illustrates an alert system method associated with a function, in accordance with an example of the present disclosure.

FIG. 16 illustrates a community level method associated with a function, in accordance with an example of the present disclosure.

FIG. 17 illustrates an exemplary process associated with machine learning module, in accordance with an example of the present disclosure

The figures depict various examples for purposes of illustration only. One skilled in the art will readily recognize from the following discussion that alternative examples of the structures and methods illustrated herein may be employed without departing from the principles described herein.

### DETAILED DESCRIPTION

Some examples of the present invention will now be described more fully hereinafter with reference to the accompanying drawings, in which some, but not all examples of the invention are shown. Indeed, various examples of the invention may be embodied in many different forms and should not be construed as limited to the examples set forth herein. Like reference numerals refer to like elements throughout. As used herein, the terms “data,” “content,” “information” and similar terms may be used interchangeably to refer to data capable of being transmitted, received or stored in accordance with examples of the invention. Moreover, the term “exemplary”, as used herein, is not provided to convey any qualitative assessment, but instead merely to convey an illustration of an example. Thus, use of any such terms should not be taken to limit the spirit and scope of examples of the invention.

As defined herein a “computer-readable storage medium,” which refers to a non-transitory, physical or tangible storage medium (e.g., volatile or non-volatile memory device), may be differentiated from a “computer-readable transmission medium,” which refers to an electromagnetic signal.

As defined herein, a “energizer,” which refers to an activity (e.g., yoga, cooking, meditation, piano, or running) that may energize a user, wherein energize may refer to an increase in energy or wellness. Energizer related alerts may include tutorials for the activity.

References in this description to “an example”, “one example”, or the like, may mean that the particular feature, function, or characteristic being described is included in at least one example of the present invention. Occurrences of such phrases in this specification do not necessarily all refer to the same example, nor are they necessarily mutually exclusive.

It is to be understood that the methods and systems described herein are not limited to specific methods, specific components, or to particular implementations. It is also to be understood that the terminology used herein is for the purpose of describing particular examples only and is not intended to be limiting.

As participation in activities is moved to Internet-based applications, such as remote work arrangements, there may be a lack of chance beneficial occurrences within groups (e.g., neighbors, coworkers, or classmates, among others) that may add some familiarity and foster relationships. Disclosed herein are methods, systems, or apparatus that may provide for an adaptive collaboration tool. The adaptive collaboration tool may facilitate collaboration between groups of users (e.g., employees) and may provide for a way to analyze and address issues associated with wellness.

Systems and methods are disclosed for generating alerts or other information, such as a recommended energizer based on an energizer profile (e.g., a subset of the user profile associated with energizers), a list of group characteristics (e.g., behavioral categories set by a group), or a set of effective energizers, among other things. Characteristics may be associated with an individual or group. In some implementations, group characteristics associated with preferences of the group in relation to one or more users' interaction with an adaptive wellness system may be used to determine whether an energizer may be recommended to help improve wellness (e.g., mood, energy level, level of engagement, or any component of the energizer profile). A machine learning module may be used to generate the alerts and other information. In an example, a machine learning module may be used to develop an energizer recommendation based on an association between the list of group characteristics, the energizer profile, or a set of effective energizers. The energizer recommendation may be presented in the form of an alert, a notification, image, video, text, email, message, or any combination thereof.

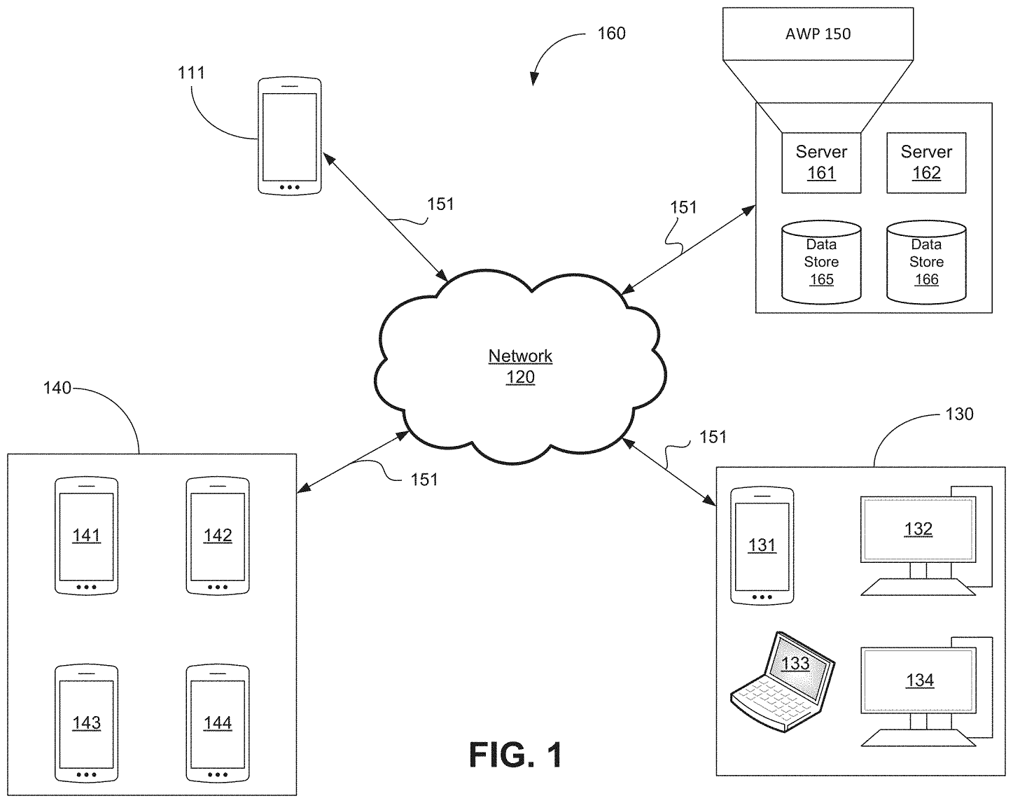

FIG. 1 illustrates an example adaptive wellness system 160 that may host an adaptive wellness platform 150 (AWP 150). The adaptive wellness system 160 may be capable of facilitating communications among entities or provisioning of content among entities. Adaptive wellness system 160 may include device 111, devices 141, 142, 143, 144 associated with group 140, devices 131, 132, 133, 134 associated with group 130, server 161, server 162, data store 165, data store 165, or AWP 150. As shown, for simplicity, AWP 150 may be located on server 161. It is contemplated that AWP 150 may be located on or interact with one or more devices of adaptive wellness system 160.

In particular examples, device 111 may be associated with an individual (e.g., a user), group 130 may be associated with an entity (e.g., an enterprise, business, or third-party application), or group 140 may be associated with a community (e.g., group of individuals or entities) that interact or communicates with AWP 150. AWP 150 may be considered a collaborative platform (or an adaptive wellness media platform or social media platform). In particular examples, one or more users may use one or more devices (e.g., device 111, 131, 132, 133, 134, 141, 142, 143, 144) to access, send data to, or receive data from AWP 150 which may be located on server 161, server 162, device (e.g., device 111, 131, 132, 133, 134, 141, 142, 143, 144), or the like.

This disclosure contemplates any suitable network 120. As an example and not by way of limitation, one or more portions of network 120 may include an ad hoc network, an intranet, an extranet, a virtual private network (VPN), a local area network (LAN), a wireless LAN (WLAN), a wide area network (WAN), a wireless WAN (WWAN), a metropolitan area network (MAN), a portion of the Internet, a portion of the Public Switched Telephone Network (PSTN), a cellular telephone network, or a combination of two or more of these. Network 120 may include one or more networks 120.

Links 151 may connect device 111, devices of group 130, devices of group 140, or adaptive wellness hub 150 to network 120 or to each other. This disclosure contemplates any suitable links 151. In particular examples, one or more links 151 include one or more wireline (such as for example Digital Subscriber Line (DSL) or Data Over Cable Service Interface Specification (DOCSIS)), wireless (such as for example Wi-Fi or Worldwide Interoperability for Microwave Access (WiMAX)), or optical (such as for example Synchronous Optical Network (SONET) or Synchronous Digital Hierarchy (SDH)) links. In particular examples, one or more links 151 each include an ad hoc network, an intranet, an extranet, a VPN, a LAN, a WLAN, a WAN, a WWAN, a MAN, a portion of the Internet, a portion of the PSTN, a cellular technology-based network, a satellite communications technology-based network, another link 151, or a combination of two or more such links 151. Links 151 need not necessarily be the same throughout network environment 100. One or more first links 151 may differ in one or more respects from one or more second links 151.

In particular examples, device 111, 131, 132, 133, 134, 141, 142, 143, 144 may be an electronic device including hardware, software, or embedded logic components or a combination of two or more such components and capable of carrying out the appropriate functionalities implemented or supported by the device 111, 131, 132, 133, 134, 141, 142, 143, 144. As an example and not by way of limitation, device 111, 131, 132, 133, 134, 141, 142, 143, 144 may be a computer system such as for example, a desktop computer, notebook or laptop computer, netbook, a tablet computer (e.g., smart tablet), e-book reader, global positioning system (GPS) device, personal digital assistant (PDA), handheld electronic device, cellular telephone, smartphone, augmented/virtual reality device, other suitable electronic device, or any suitable combination thereof. This disclosure contemplates any suitable device (e.g., device 111, 131, 132, 133, 134, 141, 142, 143, 144). A device 111, 131, 132, 133, 134, 141, 142, 143, 144 may enable a user to access network 120. A device 111, 131, 132, 133, 134, 141, 142, 143, 144 may enable a user(s) to communicate with other users at other device 111, 131, 132, 133, 134, 141, 142, 143, 144.

In particular examples, AWP 150 (also referred to herein as an adaptive wellness hub 150) may be a network-addressable computing system that can host an online wellness network. AWP 150 may generate, store, receive, or send wellness information (also referred herein as wellness data) associated with a user, such as, for example, user-profile data (e.g., energizer profile data), energy level, mood, preferred energizer, geographical location, level of engagement, or other suitable data related to the AWP 150. AWP 150 may be accessed by one or more components of adaptive wellness system 160 directly or via network 120. As an example and not by way of limitation, device 111 may access AWP 150 located on server 161 by using a web browser or a native application on device 111 associated with AWP 150 (e.g., a mobile adaptive wellness application, a messaging application, another suitable application, or any combination thereof) directly or via network 120. In particular examples, adaptive wellness system 160 may include one or more servers 161, 162. Each server 161, 162 may be a unitary server or a distributed server spanning multiple computers or multiple datacenters. Servers 161, 162 may be of various types, such as, for example and without limitation, web server, news server, mail server, message server, advertising server, file server, application server, exchange server, database server, proxy server, another server suitable for performing functions or processes described herein, or any combination thereof. In particular examples, each server 161, 162 may include hardware, software, or embedded logic components or a combination of two or more such components for carrying out the appropriate functionalities implemented or supported by server 161, 162. In particular examples, adaptive wellness system 160 may include one or more data stores 165, 166. Data stores 165, 166 may be used to store various types of information. In particular examples, the information stored in data stores 165, 166 may be organized according to specific data structures. In particular examples, each data store 165, 166 may be a relational, columnar, correlation, or other suitable database. Although this disclosure describes or illustrates particular types of databases, this disclosure contemplates any suitable types of databases. Particular examples may provide interfaces that enable device 111, 131, 132, 133, 134, 141, 142, 143, 144 or another system (e.g., a third-party system) to manage, retrieve, modify, add, or delete, the information stored in data store 165, 166.

In particular examples, AWP 150 may store one or more energizer profiles in one or more data store 165, 166. In particular examples, an energizer profile may include multiple nodes—which may include multiple user nodes (each corresponding to a particular user associated with a device 111) or multiple concept nodes (each corresponding to a particular role or concept)—and multiple edges connecting the nodes. Users of the AWP 150 may have the ability to communicate and interact with other users. In particular examples, users associated with a particular company (e.g., group 130) may join the AWP 150 and then add connections (e.g., relationships) to a number of other users of the same particular group 130 constituting a group (e.g., group 140) of AWP 150 to whom they want to be connected. User connections or communications may be monitored. In an example, server 161 of adaptive wellness hub 150 may receive, record, or otherwise obtain information associated with communications or connections of users (e.g., device 111, device 131, etc.). Herein, the term trusted member (e.g., trusted co-worker or club of friends) may refer to any other user of AWP 150 in which there is indication of a connection or relationship.

In particular examples, AWP 150 may provide users with the ability to take actions on various types of items. As an example and not by way of limitation, the items may include groups (e.g., group 140) to which a user may belong, messaging boards in which a user might be interested, question forums, interactions with images or videos, instructions on how to perform actions associated with a user's job or role, or other suitable items. A user may interact with anything that is capable of being represented in AWP 150. A user may be assigned a role, such as team champion based on an analysis of engagement on AWP 150. Such roles may be assigned different weights in determining user or group engagement, user or group mood, or other user or group information associated with AWP 150.

In particular examples, AWP 150 may be capable of linking a variety of entities. As an example and not by way of limitation, AWP 150 may enable users to interact with each other as well as receive content from their respective group 130 or other entities, or to allow users to interact with these entities through an application programming interfaces (API) or other communication channels.

AWP 150 may include user-generated content objects, which may enhance a user's interactions with AWP 150. User-generated content may include any data a user may add, upload, send, or “post” that is made available publicly or privately, to a specific group or community, to AWP 150. As an example and not by way of limitation, a user may communicate posts to AWP 150 from a device 111. Posts may include data such as questions, answers to questions, or other textual data, photos, videos, audio, links, or other similar data or media associated with the group of users that is available to AWP 150.

Although FIG. 1 illustrates a particular arrangement of device 111, network 120, devices of group 130, devices of group 140, server 161, data store 165, or AWP 150, among other things, this disclosure contemplates any suitable arrangement. The devices of adaptive wellness system 160 may be physically or logically co-located with each other in whole or in part.

In network environment 100, the adaptive wellness system 160 may receive user specific information, via network 120, link 151, or any other suitable means, through a device (e.g., device 111). For example, AWP 150 may receive a first users' data (e.g., via device 111) indicating the first user's wellness state, as well as data indicating a desired wellness state, or other wellness information. In some examples, the desired wellness state may be determined via user's preference, a company (e.g., group 130) preferences (e.g., a global company set preference), AWP 150, or other systems (e.g., collaborative information environment system or connected watches, etc.). While specific devices may be shown in FIG. 1, AWP 150 may receive data from any type of device including a computer, smart tablet, smart phone, or the like. In addition to receiving information from users, the AWP 150 may also receive information form one or more devices of one or more groups (e.g., group 130). For example, group 130 may have several employees (e.g., employees associated with device 131, 132, 133, 134) that may make up a sub-group of the company. AWP 150 may also receive information from each member of a group 140 (e.g., users associated with device 141, 142, 143, 144) relating to both their current, and desired wellness state, which may be determined via each user's preference, a company (e.g., group 130) associated with the users (e.g., a global company set preference), or the AWP 150. In some examples, information received, via AWP 150, may enable a company (e.g., group 130) to obtain an understanding of the overall wellness of the group (e.g., group 130). With information on the current or desired states for individuals or groups, the adaptive wellness system may provide energizer recommendations to devices of one or more user that may help users reach a respective preferred wellness state, which may adjust the overall wellness of groups 140 (e.g., a community) to which they belong as well as the work environment associated with the users.

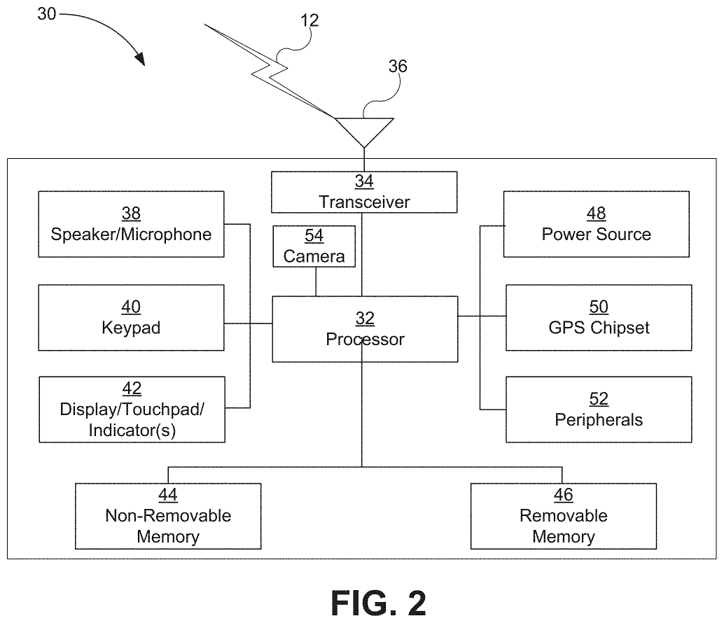

FIG. 2 illustrates a block diagram of an example hardware/software architecture of a device such as, for example, user equipment (UE) 30. In some examples, the UE 30 may be any of device 111, 131, 132, 133, 134, 141, 142, 143, 144. In some examples, the UE 30 may be a computer system such as for example a desktop computer, notebook or laptop computer, netbook, a tablet computer (e.g., a smart tablet), e-book reader, GPS device, camera, personal digital assistant, handheld electronic device, cellular telephone, smartphone, smart glasses, augmented/virtual reality device, smart watch, or any other suitable electronic device. As shown in FIG. 2, UE 30 (also referred to herein as node 30) may include a processor 32, non-removable memory 44, removable memory 46, a speaker/microphone 38, a keypad 40, a display, touchpad, or indicators 42, a power source 48, a global positioning system (GPS) chipset 50, and other peripherals 52. The power source 48 may be capable of receiving electric power for supplying electric power to the UE 30. For example, the power source 48 may include an alternating current to direct current (AC-to-DC) converter allowing the power source 48 to be connected/plugged to an AC electrical receptable or Universal Serial Bus (USB) port for receiving electric power. The UE 30 may also include a camera 54. In an example, the camera 54 may be a smart camera configured to sense images/video appearing within one or more bounding boxes. The one or more cameras 54 may capture one or more images/videos indicative of a scene (e.g., from a viewpoint of a user). In other words, the one or more cameras 54 may identify/capture the scene or view which the user sees. The UE 30 may also include communication circuitry, such as a transceiver 34 and a transmit/receive element 36. It will be appreciated the UE 30 may include any sub-combination of the foregoing elements while remaining consistent with an example.

The processor 32 may be a special purpose processor, a digital signal processor (DSP), a plurality of microprocessors, one or more microprocessors in association with a DSP core, a controller, a microcontroller, Application Specific Integrated Circuits (ASICs), Field Programmable Gate Array (FPGAs) circuits, any other type of integrated circuit (IC), a state machine, and the like. In general, the processor 32 may execute computer-executable instructions stored in the memory (e.g., non-removable memory 44 or removable memory 46) of the node 30 in order to perform the various required functions of the node. For example, the processor 32 may perform signal coding, data processing, power control, input/output processing, or any other functionality that enables the node 30 to operate in a wireless or wired environment. The processor 32 may run application-layer programs (e.g., browsers) or radio access-layer (RAN) programs or other communications programs. The processor 32 may also perform security operations such as authentication, security key agreement, or cryptographic operations, such as at the access-layer or application layer for example.

The processor 32 is coupled to its communication circuitry (e.g., transceiver 34 and transmit/receive element 36). The processor 32, through the execution of computer executable instructions, may control the communication circuitry in order to cause the node 30 to communicate with other nodes via the network to which it is connected.

The transmit/receive element 36 may be configured to transmit signals to, or receive signals from, other nodes or networking equipment. For example, in an example, the transmit/receive element 36 may be an antenna configured to transmit or receive radio frequency (RF) signals. The transmit/receive element 36 may support various networks and air interfaces, such as wireless local area network (WLAN), wireless personal area network (WPAN), cellular, and the like. In yet another example, the transmit/receive element 36 may be configured to transmit or receive both RF and light signals. It will be appreciated that the transmit/receive element 36 may be configured to transmit or receive any combination of wireless or wired signals.

The transceiver 34 may be configured to modulate the signals that are to be transmitted by the transmit/receive element 36 and to demodulate the signals that are received by the transmit/receive element 36. As noted herein, the node 30 may have multi-mode capabilities. Thus, the transceiver 34 may include multiple transceivers for enabling the node 30 to communicate via multiple radio access technologies (RATs), such as universal terrestrial radio access (UTRA) and Institute of Electrical and Electronics Engineers (IEEE 802.11), for example.

The processor 32 may access information from, and store data in, any type of suitable memory, such as the non-removable memory 44 or the removable memory 46. For example, the processor 32 may store session context in its memory, as described herein. The non-removable memory 44 may include RAM, ROM, a hard disk, or any other type of memory storage device. The removable memory 46 may include a subscriber identity module (SIM) card, a memory stick, a secure digital (SD) memory card, and the like. In other examples, the processor 32 may access information from, and store data in, memory that is not physically located on the node 30, such as on a server or a home computer.

The processor 32 may receive power from the power source 48, and may be configured to distribute or control the power to the other components in the node 30. The power source 48 may be any suitable device for powering the node 30. For example, the power source 48 may include one or more dry cell batteries (e.g., nickel-cadmium (NiCd), nickel-zinc (NiZn), nickel metal hydride (NiMH), lithium-ion (Li-ion), etc.), solar cells, fuel cells, and the like. The processor 32 may also be coupled to the GPS chipset 50, which may be configured to provide location information (e.g., longitude and latitude) regarding the current location of the node 30. It will be appreciated that the node 30 may acquire location information by way of any suitable location-determination method while remaining consistent with an example.

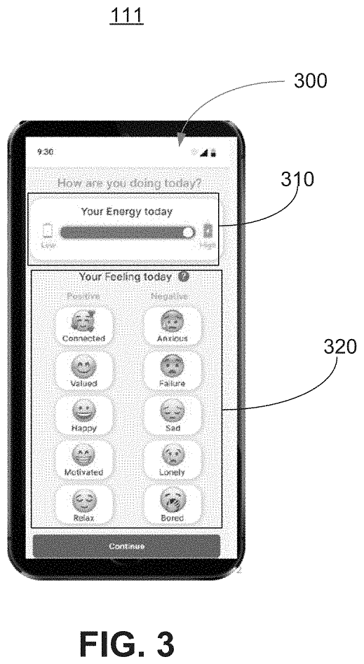

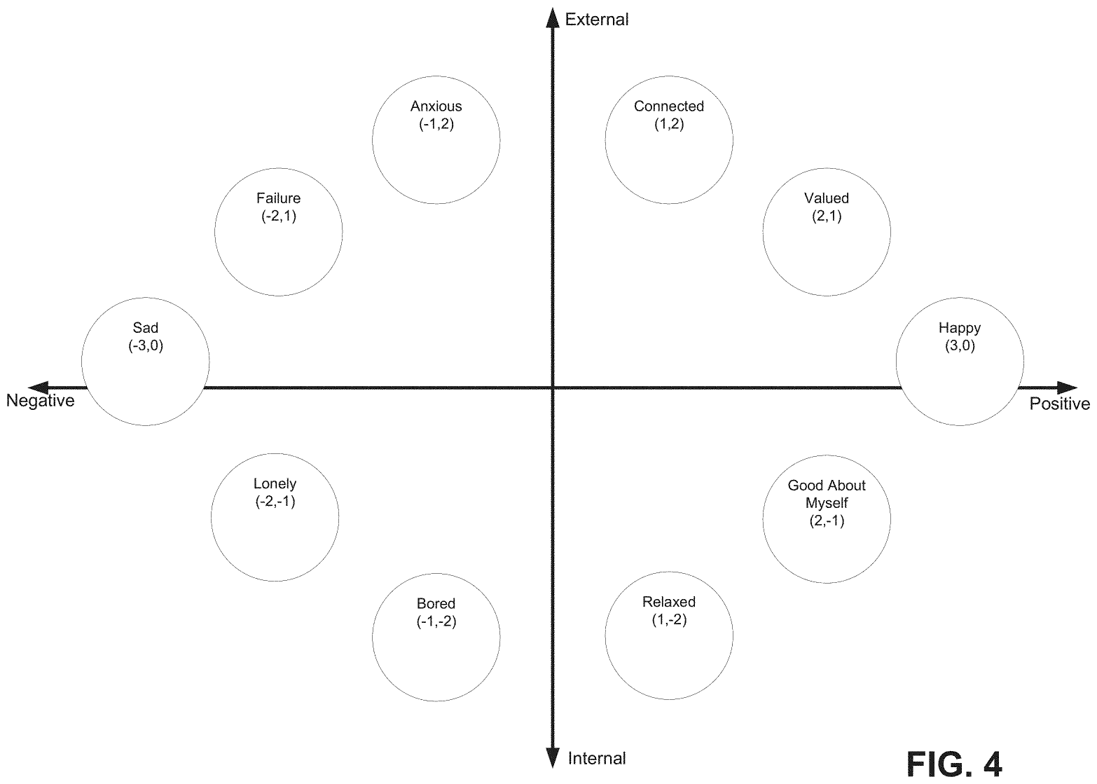

FIG. 3 illustrates an example user interface 300 for an adaptive wellness platform (e.g., AWP 150), in accordance with an example of the present disclosure. User interface 300 of a device (e.g., device 111) associated with a user may include areas for the user to indicate attributes of the user's wellness, which may be during a workday. User interface 300 may prompt (e.g., display, provide haptic feedback, etc.) a user to indicate their mood via mood area 320 where a user is displayed emojis to express their mood, and their energy level via energy area 310. Emojis may refer to digital pictograms used widely throughout communication between users via social media, text, email, and computer-mediated communications to express emotions. The emojis may link to specific indications of mood, psychological health, or feelings of wellness, which is described by Jason Davies, Mark McKenna, Kate Denner, Jon Bayley, and Matthew Morgan in the journal article The Emoji Current Mood and Experience Scale: The Development and Initial Validation of an Ultra-Brief Literacy Independent Measure of Psychological Health published in the Journal of Mental Health, which is incorporated by reference in its entirety. As shown in FIG. 4, with reference to the journal article by Jason Davies et al. vectors may be created to pair a user's mood indicated via emoji with a calculable value illustrated by a vector (e.g., (‘x’, ‘y’)), where a positive ‘x’ value of the vectors may be representative of a user having a positive feeling or mood and a negative ‘x’ value may represent a user having a negative feeling or mood.

Furthermore, the ‘y’ value of the vectors may represent a user's feeling of connectedness with their environment, work, members (e.g., co-workers), or trusted members, where a negative ‘y’ value represents a user who is feeling internal connectedness meaning that their level of connectedness with their work, environment, members, or trusted members may be low and is more likely to not be engaged in communications with other users of adaptive wellness system 160. Conversely, where the vector ‘y’ value is positive, a user may be feeling externally connected to their work, environment, members, or trusted members, meaning that the user may feel connected to their work, environment, members, or trusted members and is more likely to be engaged in communications with other users of adaptive wellness system 160. The calculable values of mood illustrated by vectors may be any combination of values weighted based on behavioral categories determined via a company (e.g., group 130) associated with the user. The behavioral categories may include one or more behavioral models, such as 5R Behavioral Model described by Cecile Dejoux in Métamorphose des managers à l'ère du numérique et de l'intelligence artificielle published in 2018, which is incorporated by reference in its entirety. 5R may refer to the roles, rules, respect, recognition, and routines of a group 130 where they may define what each of these behavioral categories mean for their constituents. Based on the hierarchy or meaning of the 5R's determined by the company (e.g., group 130), the vectors values associated with mood may be changed respectively to achieve the goals associated with the group 130.

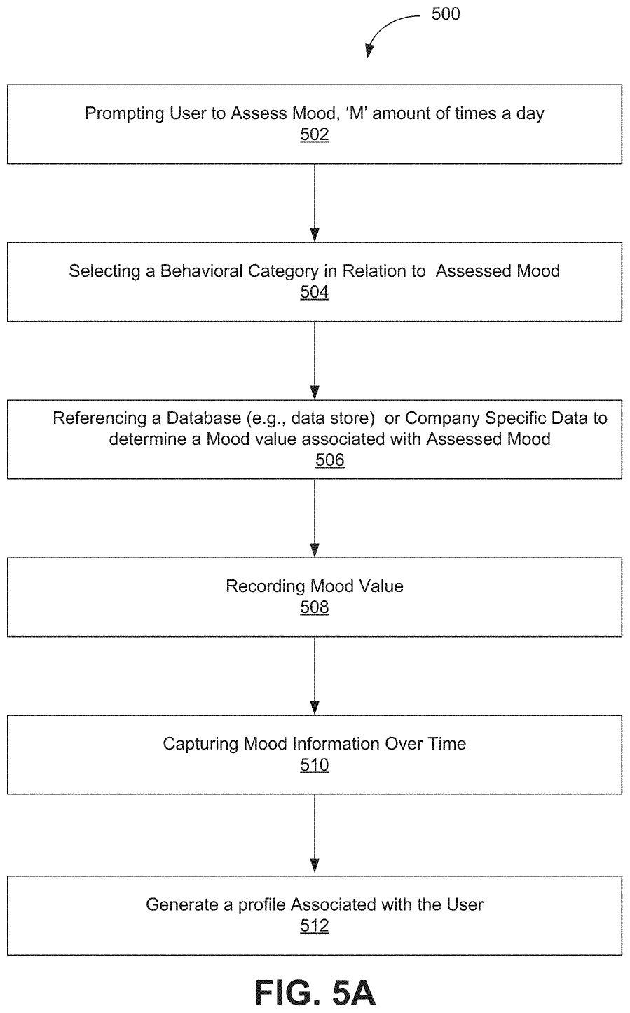

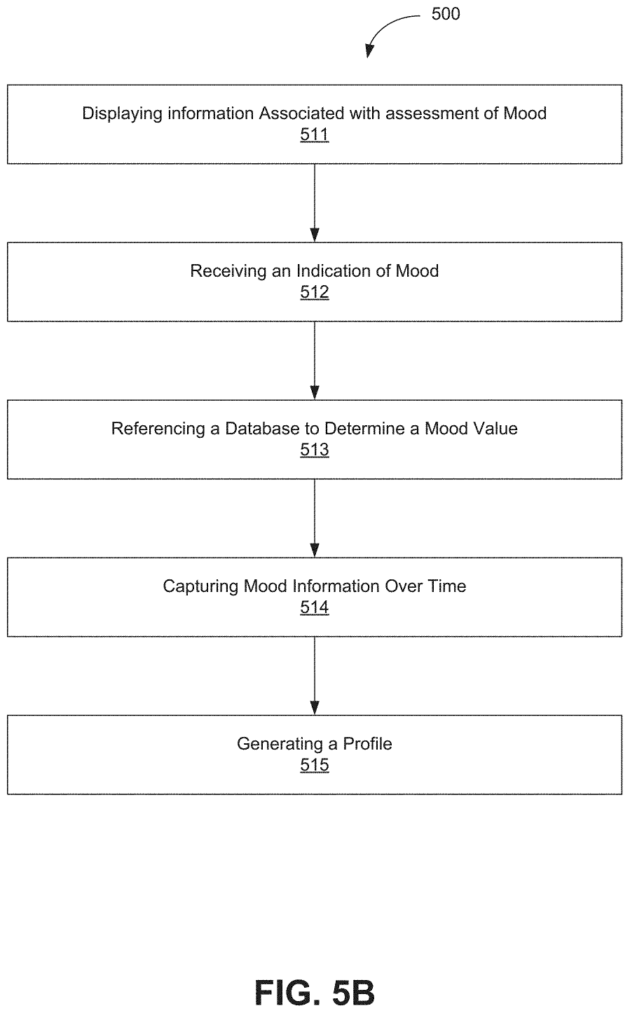

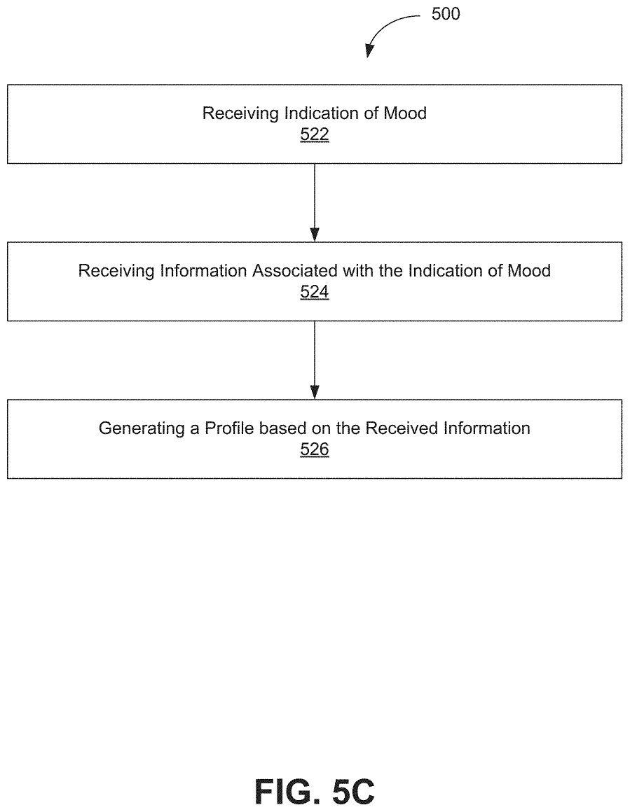

FIG. 5A illustrates an example process of mood capture associated with a user. A mood capture process 500 associated with a user may include prompting a user to assess their mood in an instance where the user assesses the adaptive wellness platform (e.g., AWP 150); selecting a behavioral category in relation to assessed mood at that time; referencing a data base (e.g., data store 165, 166) or company specific data determined via company (e.g., group 130) and stored in a data base (e.g., data store 165, 166, non-removable memory 44, or removable memory 46) of adaptive wellness system) to determine a mood value or associated with assessed mood; recording mood value for that time on a given day indicated via user interface (e.g., display/touchpad/indicator(s) 42); recording mood value; and capturing mood information over time to formulate a profile associated with the user. Process 500 is further described herein.

At step 502, a prompt may be sent by a device (e.g., device 111). The prompt may be sent to the user via user interface 300 of device 111 and may request information associated with assessing the mood of the user associated with device 111. In some examples, the user may be prompted to assess their mood ‘M’ number of times a day (or some other indicated period of time) depending on a group's (e.g., group 130) preference. At step 504, the device 111 may receive, via user interface 300 of the display (e.g., display/touchpad/indicator(s) 42) or other input actions (e.g., voice input), an indication of mood via selection of an emoji on the user interface 300 of the device 111 in relation to assessed mood at that time. At step 506, adaptive wellness system 160 may communicate with a database (e.g., data store 165, 166) or company (e.g., group 130) specific data stored via data store 165, 166 to obtain a mood value associated with the assessed mood, wherein the mood value may be a weighted vector associated with a behavioral category. It is contemplated that other wellness information may be a calculable value illustrated by a vector. In some examples, company specific data may be stored in a memory (e.g., non-removable memory 44 or removable memory 46) of a company (e.g., group 130) or user device (e.g., device 111).

At step 508, device 111 may record mood value and information associated with the mood of the user (also referred herein as mood information). Mood information may include the recorded mood value, time, day of the week, date, geographical location associated with the user, or any other suitable information associated with the mood. At step 510, mood information may be stored in a database or memory of the device 111 (e.g., non-removable memory 44 or removable memory 46). At step 512, a profile (also referred to herein as an energizer profile) associated with the user may be generated (e.g., updated) based on the mood information, among other things as disclosed herein. In some examples the mood information may be stored in a database of adaptive wellness system 160 (e.g., data store 165, 166). In some particular examples, mood information may be stored hourly, daily, weekly, monthly, every six months, or yearly to create a time dependent profile associated with the user to monitor how a user's mood may change over time, this profile may also be referred to or comprise an energizer profile. Although FIG. 5 illustrates mood, the mood capture process 500 may be utilized to determine any suitable variable associated with wellness such as, engagement level.

FIG. 5B illustrates an example mood capture process 500 associated with a user. At step 511, device 111 may display information associated with assessing a mood of a user via display (e.g., display/touchpad/indicator(s) 42) of device 111. At step 512, device 111 may receive an indication of mood of the user. The indication may be based on receiving a selection on a display (e.g., display/touchpad/indicator(s) 42) of device 111, voice input, or other suitable input. At step 513, based on the indication of step 512, device 111 may reference a database (e.g., data store 161.162) of AWP 150 to determine a mood value, which may be weighted based on behavioral categories determined by a company (e.g., group 130). At step 514, device 111 may capture mood information associated with the indicated mood such as recorded mood value, time, day of the week, date, geographical location associated with the user, or any other suitable information associated with the mood. At Step 515, device 111 may generate a profile (e.g., energizer profile) associated with the user.

FIG. 5C illustrates an example mood capture process 500 associated with a user. At step 522, a server (e.g., server 161) of the AWP 150 may receive an indication of the mood of a user(s). At step 524, server 161 may receive information (e.g., time, date, or location) associated with the indication of mood. Location may be further broken down to work, home, or geographical coordinates. Times may be associated with work time period, commute time period, home time period, or any other time suitable time period. At step 526, server 161 may generate a profile (e.g., energizer profile) associated with the user.

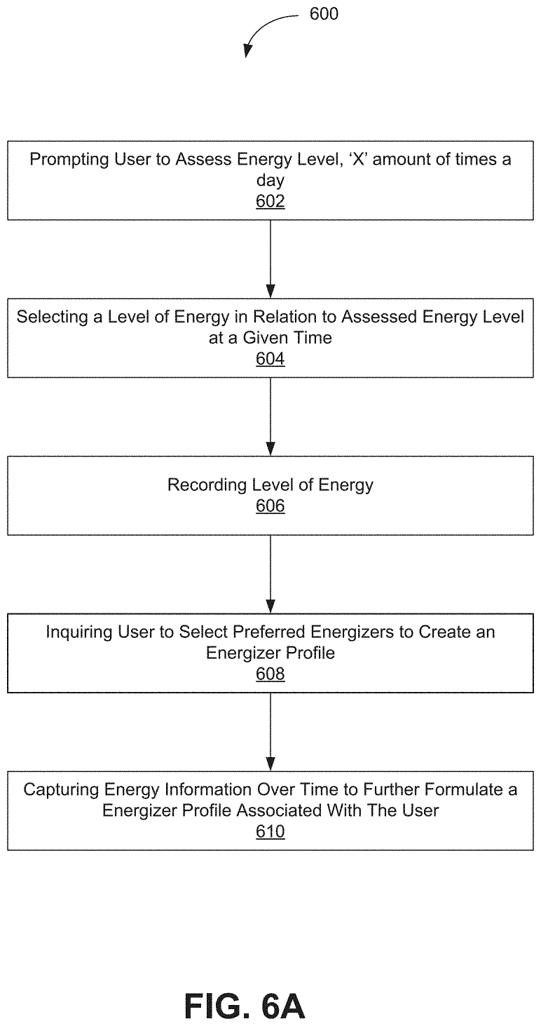

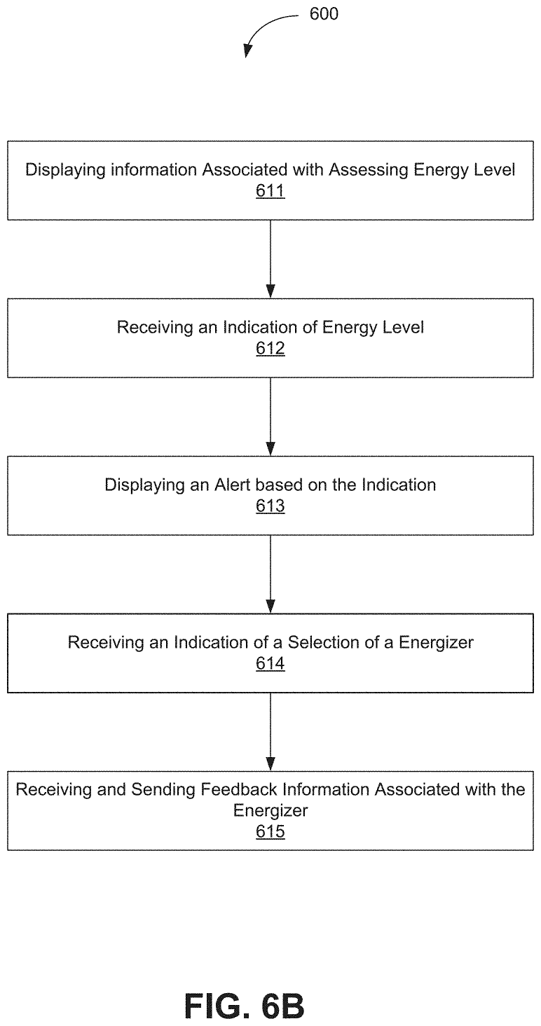

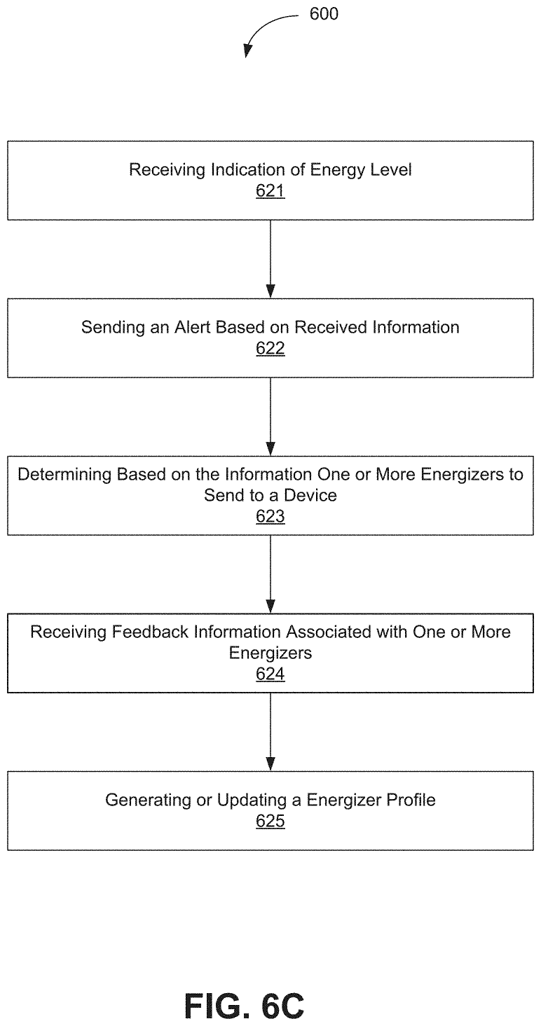

FIG. 6 illustrates an example process 600 of energy level capture associated with a user. Process 600 may include prompting a user to assess their energy level in an instance where the user assesses the adaptive wellness platform (e.g., AWP 150); selecting a level of energy in relation to assessed energy level at that time; recording level of energy for that time on a given day indicated via user interface (e.g., display/touchpad/indicator(s) 42); inquiring user to select from a series of energizers, via display of the device, preferred energizers in a given setting or environment (e.g., work or home) to create an energizer profile associated with the user; capturing energy information over time to further formulate a profile associated with the user (e.g., profile shown in step 510). Process 600 is further described herein.

At step 602, a device (e.g., device 111) may prompt a user to assess their energy level, via user interface (e.g., user interface 300). In some examples, the user may be prompted to assess their energy level ‘M’ number of times a day depending on a company's (e.g., group 130) preference. At step 604, a user may select, via user interface 300 of the display (e.g., display/touchpad/indicator(s) 42), a level of energy in relation to assessed energy level at that time, where the energy level selected may correspond to any suitable number range such as, for example, the number range between zero and ten, where ten may correspond to maximum energy level and 0 may correspond to minimum energy level.

At step 606, device 111 may record level of energy and information associated with the energy level of the user, such as time, day of the week, date, location associated with the user, or any other suitable information associated with the energy level. The level of energy and information associated with the energy level of the user may be stored in a database or memory the device 111 (e.g., non-removable memory 44 or removable memory 46) over time to formulate a profile associated with the user. In some examples, the energy level information may be stored in a database of adaptive wellness system 160 (e.g., data store 166, 166). In some particular examples, mood information may be stored weekly, monthly, every six months, or yearly to create a mood profile associated with the user.

At step 608, AWP 150 may communicate to device 111 via network 120 to inquire user to select preferred energizers out of a list of energizers to create an energizer profile associated with the user. In some examples, the list of energizers may be categorized by location, if a user is in a work location or at another location such as, the user's home. In some particular examples, the list of energizers for a first location (e.g., work) may be shorter in length of time to complete than the energizers in other locations. Therefore, each location may have a different amount of time allotted. In some other examples, the user may be prompted to provide their preferred energizers from the list of energizers relative to their location at that moment, for example, if a user is at their work location the list of energizers may only comprise energizers suitable for the work environment. At step 610, energy level information and preferred energizers may be stored in a database or memory of the device 111 (e.g., non-removable memory 44 or removable memory 46) over time to further formulate a profile associated with the user in conjunction with the mood information of step 510 of FIG. 5. In some examples the energy level information may be stored in a database of adaptive wellness system 160 (e.g., data store 166, 166). In some particular examples, energy level information may be stored weekly, monthly, every six months, or yearly to create an energizer profile associated with the user to monitor how a user's energy level may change over time, as well as compile preferred energizers associated with the users. The processes of FIG. 5 and FIG. 6 may occur in no particular order, concurrently, simultaneously, sequential, or any combination thereof.

FIG. 6B illustrates an example mood capture process 600 associated with a user. At step 611, device 111 may display information associated with assessing an energy level of a user via display (e.g., display/touchpad/indicator(s) 42) of device 111. At step 612, device 111 may receive an indication of the energy level of the user. The indication may be based on receiving a selection on a display (e.g., display/touchpad/indicator(s) 42) of device 111, voice input, or other suitable input. At step 613, based on the indication of step 612 or other information (e.g., mood, level of engagement, or any other suitable information or any combination thereof), device 111 may send (e.g., display) an alert. The alert may be associated with one or more energizers. As disclosed in more detail herein, the alert may be determined based on an assessment of wellness information (e.g., mood, energy level, location, level of engagement, or any other suitable information or any combination thereof) or the use of machine learning. Wellness information may include key behavior indicators, such as individual mood, group mood, role engagement (e.g., leader or contributor), routine engagement, collaborative engagement, or personal activity.

At step 614, device 111 may receive an indication of a selection of a first energizer (e.g., a first preferred energizer) of a list of one or more energizers, which may have been incorporated in the alert of step 613. The indication may be based on receiving a selection on a display of device 111, voice input, or any other suitable input. At step 615, device 111 may receive or send feedback information associated with the first energizer to a server (e.g., server 161). The feedback information may be an indication of the effectiveness of the first energizer to increase wellness (e.g., mood, energy level, level of engagement, or any combination thereof) associated with the user. The indication may be from a user selection or analysis of user-related subsequent actions associated with AWP 150. Subsequent actions may include user interactions with content posted, published, or otherwise interacted with, within a particular date range that may be extracted from a user profile or energizer profile. Interactions may include, but are not limited to a like, view, post, comment, or other user action with respect to the content. In some examples, server 161 may be a part of AWP 150, where it may run an analysis on one or more users and send or receive information based on the inputs of the users.

FIG. 6C illustrates an example mood capture process 600 associated with a user. At step 621, a server (e.g., server 161) of the AWP 150 may receive an indication of an energy level of a user(s). At step 622, based on the indication, the server 161 may send an alert to device 111 associated with the user. The alert may be associated with one or more energizers. As disclosed in more detail herein, the alert may be determined based on an assessment of wellness information (e.g., mood, energy level, location, level of engagement, or any other suitable information or any combination thereof) or the use of machine learning operations (also referred to as algorithms or modules herein). At step 623, further based on the received indication of step 621, the server 161 may determine, based on the information received, one or more energizers to be sent to the device 111 associated with a user. At step 624, server 161 may receive feedback information associated with a first energizer (e.g., a first preferred energizer) selected via step 614 of FIG. 6B. Feedback information may include differences in information (e.g., mood, energy level, level of engagement, or any combination thereof) attributed to performance of an energizer. At step 625, server 161 may generate or update the profile (e.g., energizer profile) based on the feedback information. It is to be contemplated that the steps of FIGS. 5A, 5B, and 5C and FIGS. 6A, 6B, and 6C may occur on different devices or be distributed across multiple devices.

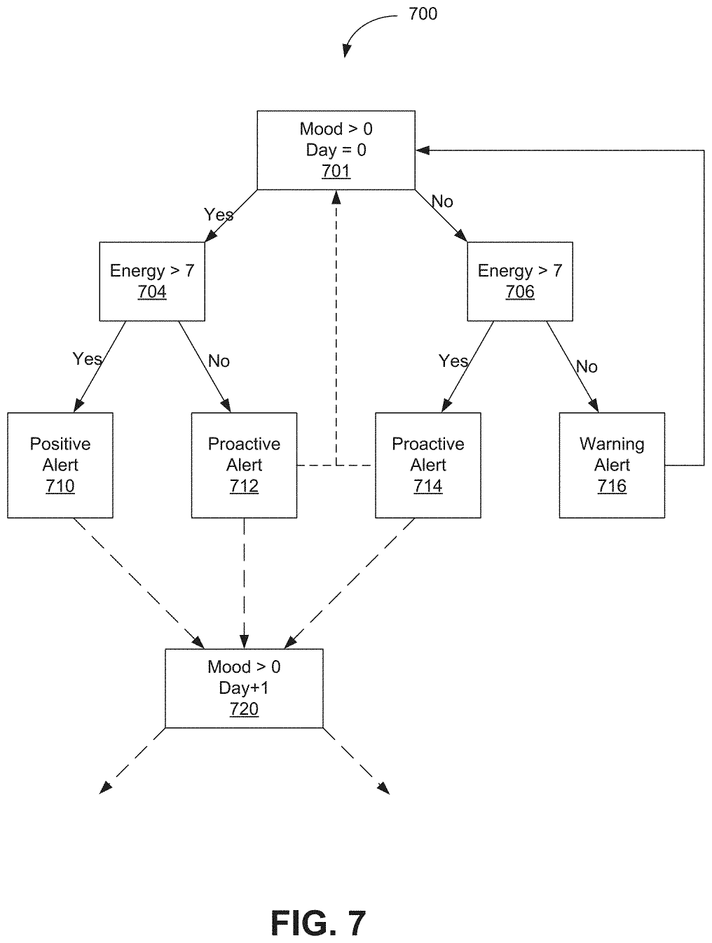

FIG. 7 illustrates an example alert process associated with mood and energy level of a user. The alert process 700 to alert a user, company member, or any other suitable member of a group 130 (e.g., company) or organization may include assessing mood on a given day, denoted as day=0; assessing the energy level associated with the user; determining based on energy level to send a notification or message to the user. An alert may be a pop-up alert, post on a user feed, a message, email, or any other suitable form of alert or some combination thereof, depending on a user, or a company's (e.g., group 130) preference. The alert may similarly be presented by a device (e.g., device 111, 131, 132, 133, 134, 141, 142, 143, 144) in a variety of forms to a user's device 111, such as text or banner on a home screen, an alert or notification within an app, when interacting with the adaptive wellness platform (e.g., AWP 150), or the like. Process 700 is further described herein. Using machine learning, the decision tree disclosed herein, including thresholds or sequences, may evolve with training data.

At block 701, mood information captured via process 500 may be assessed at a given day. For example, if a first user and second user indicated on Monday (i.e., day=0) that their mood level is happy, the mood associated with the first user and second user is positive, as shown in FIG. 4, where mood may be determined via process 500 of FIG. 5. In this example, the mood is greater than zero for both the first user associated with device 111 and the second user associated with device 141, and the process 700 may proceed to block 704. At block 704, energy level information captured, via process 600 as shown in FIG. 6, may be referenced, and used to determine whether a threshold is met to proceed to block 710 or 712. Continuing with the previous example where the first user and the second user indicated on Monday that their mood was happy, the first user also indicated that their energy level was ten corresponding to a maximum energy level, via process 600 of FIG. 6. Thus, the process 700 may proceed to block 710, where the user may receive a positive alert, via display of the user device (e.g., device 111). A positive alert, may provide text to the first user such as, “You feel great. Would you like to test new Energizers to improve their efficiency?” or any other suitable text determined via server (e.g., server 161, 162) of adaptive wellness system 160.

Conversely, in the previous example, the second user may have indicated that their energy level is zero corresponding to a minimum energy level, via process 600 of FIG. 6. Therefore, in this particular example, the process 700 may proceed to block 712, where the user may receive a proactive alert, via the display of the user device (e.g., device 111). In this example, a proactive alert may be used to aid the second user's energy level before the second user's energy level affects their overall wellness by providing a proactive alert. The proactive alert may provide text to the second user such as, “We invite you to increase your energizer activity”, or any other suitable text determined via server (e.g., server 161, 162) of adaptive wellness system 160.

At block 710 and 712, an alert may be provided to the user. The alert may suggest an energizer be performed by the user, where the user may decide whether they want to perform the energizer. At block 720, it is contemplated that the day is changed and the process of 700 may be repeated for the next day (or another indicated period) that the user accessed the AWP 150.

Alternatively, at block 701, mood information captured via process 500 may be assessed at a given day (or another indicated period). In this example, if a first user and second user indicated on Monday (i.e., day=0) that their mood level is sad, the mood associated with the first user and second user is negative, as shown in FIG. 4, where mood may be determined via process 500 of FIG. 5. In this example, the mood is less than zero for both the first user and the second user, and the process 700 may proceed to block 706. At block 706, energy level information captured, via process 600 as shown in FIG. 6, may be referenced, and used to determine whether to proceed to block 714 or 716. Continuing with the previous example where the first user and the second user indicated on Monday that their mood was sad, the first user also indicated that their energy level was ten corresponding to a maximum energy level, via process 600 of FIG. 6. Thus, the process 700 may proceed to block 714, where the user may receive a proactive alert, via display of the user device (e.g., device 111). The proactive alert may provide text to the first user such as, “We invite you to increase your energizer activity?” or any other suitable text determined via server (e.g., server 161, 162) of adaptive wellness system 160. From block 714, the process 700 may proceed to block 720 when the user accesses the AWP 150 another day, where the process 700 may start over for the new day.

Conversely, in the previous example, the second user may have indicated that their energy level is 0 corresponding to a minimum energy level, via process 600 of FIG. 6. Therefore, in this particular example, the process 700 may proceed to block 716, where the user may receive a warning alert, via the display of the user device (e.g., device 111). In this example, a warning alert may be used to aid the second user's overall wellness level, comprised of a combination of mood and energy level, before the second user's overall wellness is affected. The warning alert may provide text to the second user such as, “We invite you to take care about yourself, practice energizers diligently and speak to your friends”, or any other suitable text determined via server (e.g., server 161, 162) of adaptive wellness system 160. In some examples the alert process 700, may be iterative after block 716 within the same day to ensure that the user's wellness has improved.

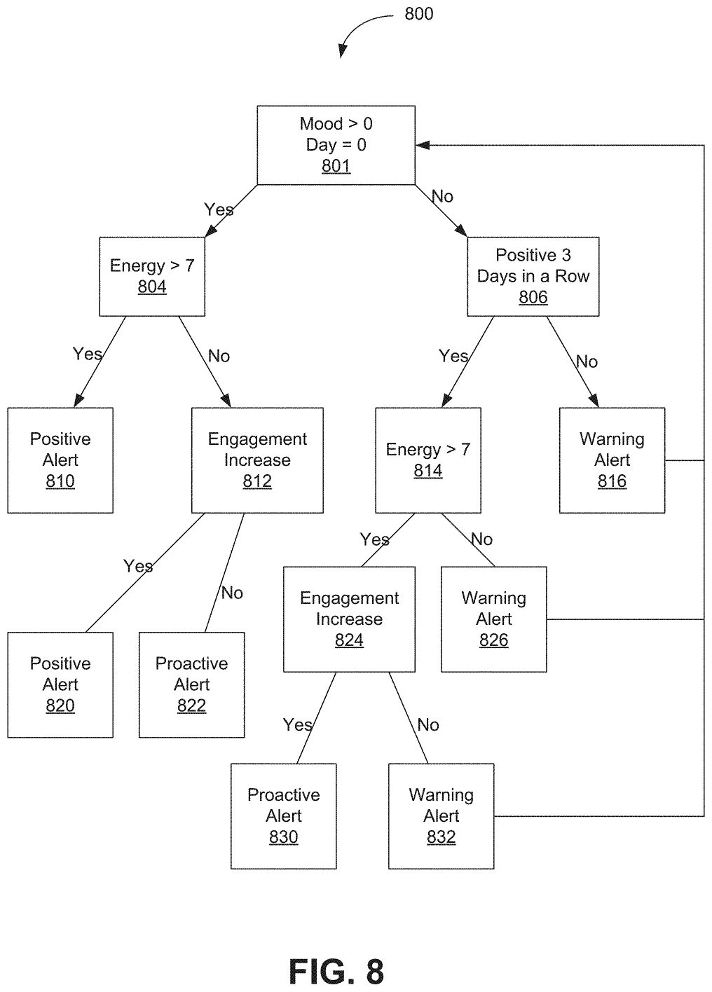

FIG. 8 illustrates an alternate example alert process associated with mood, energy level, and engagement level of a user in accordance with an example of the present disclosure. The alert process 800 to alert a user, company member, or any other suitable member of a company (e.g., group 130) or organization may include assessing mood on a given day, denoted as day=0; assessing the energy level associated with the user; or determining based on energy level to send a notification or message to the user. An alert may be a pop-up alert, notification a post on a user feed, a message, email or any other form suitable form of alert or some combination thereof, depending on a user, or a company's (e.g., group 130) preference. The alert may similarly be presented by a device (e.g., device 111, 131, 132, 133, 134, 141, 142, 143, 144) in a variety of forms to a user's device 111, such as text or banner on a home screen, an alert or notification within an app, when interacting with the AWP 150, and the like. Process 800 is further described below. Using machine learning, the decision tree disclosed herein, including thresholds or sequences, may evolve with training data.

At block 801, mood information captured via process 500 may be assessed at a given day. For example, if a first user and second user indicated on Monday (i.e., day=0) that their mood level is happy, the mood associated with the first user and second user is positive, as shown in FIG. 4, where mood is determined via process 500 of FIG. 5. In this example, the mood is greater than zero for both the first user and the second user, and the process 800 may proceed to block 804. At block 804, energy level information captured, via process 600 as shown in FIG. 6, may be referenced, and used to determine whether to proceed to block 810 or 812. Continuing with the previous example where the first user and the second user indicated on Monday that their mood was happy, the first user also indicated that their energy level was ten corresponding to a maximum energy level, via process 600 of FIG. 6. Thus, the process 800 may proceed to block 810, where the user may receive a positive alert, via display of the user device (e.g., device 111). A positive alert, may provide text to the first user such as, “You feel great. Would you like to test new Energizers to improve their efficiency?” or any other suitable text determined via server (e.g., server 161, 162) of adaptive wellness system 160.

Conversely, in the previous example, the second user may have indicated that their energy level is 0 corresponding to a minimum energy level, via process 600 of FIG. 6. Therefore, in this particular example, the process 800 may proceed to block 812, where the user may receive an alert, via the display of the user device (e.g., device 111). In this example, the alert may suggest that the second user increase their engagement within the adaptive wellness platform (e.g., AWP 150). The user's engagement may be monitored via interactions with user-generated content that may include any data a user may add, upload, send, interact with, or “post” that is made available publicly or privately, to a specific group or community, to AWP 150. Posts may include data such as questions, answers to questions, or other textual data, photos, videos, audio, links, or other similar data or media associated with the company, job, interests, or any other suitable association of the users that is available to AWP 150. In some other examples, the AWP 150 may monitor the level of engagement of the second user over time to see if the second users engagement level increased within the AWP 150. In various examples, the level of engagement (e.g., role engagement, routine engagement, or collaborative engagement) may be determined based on content posted (e.g., number or types of tips to co-workers, asks, or shares), comments (e.g., number or types of written reactions to other user posts), or people first contribution. A people first contribution may be a post, comment, or the like that contributes to engagement or wellbeing. This may be determined based on the number of likes or other indicators associated with the contribution.

At steps 820 and 822, a particular alert, positive alert, or proactive alert respectively, may be presented to a user depending on whether their engagement level has increased that day. For instances where the user engagement level increases the user may receive a positive alert. Conversely, for instances where engagement level does not increase the user may receive a proactive alert. In some examples the AWP 150 may reference user level of engagement over a period of time to determine increase of engagement.

Alternatively, at block 801, mood information captured via process 500 may be assessed at a given day. In this example, if a first user and second user indicated on Monday (i.e., day=0) that their mood level is sad, the mood associated with the first user and second user is negative, as shown in FIG. 4, where mood is determined via process 500 of FIG. 5. In this example, the mood is less than zero for both the first user and the second user, and the process 800 may proceed to block 806. At block 806, mood information captured over time, via process 500 as shown in FIG. 5, may be referenced, and used to determine whether users had a positive mood such as, connected, valued, happy, good about myself, relax, or any suitable term to define mood, for consecutive days prior, where the limit of consecutive days may be determined via company (e.g., group 130). The number of suitable consecutive days may be determined via company (e.g., group 130) preferences or determined via adaptive wellness platform 160.

Further in the example described above, the first user may have also had three positive moods in row. In this example, the alert process 800 may proceed to step 814, where energy level information may be referenced, captured via process 600 as shown in FIG. 6, and used to determine whether to proceed to block 824 or 826. Conversely, if the second user did not have consecutive days with a positive mood, thus the process 800 may proceed to step 816, where the second user may receive a warning alert.

Continuing with the previous example, where the first user and the second user indicated on Monday that their mood was sad, the first user had three days with positive moods in a row, the first user may have also indicated that their energy level was ten corresponding to a maximum energy level, via process 600 of FIG. 6. Thus, the process 800 may proceed to block 824, where the user may receive a proactive alert, via display of the user device (e.g., device 111). Conversely, if the first user had indicated that their energy level was 0 corresponding to a low energy level at step 814, the process 800 may proceed to block 826, where the first user may receive a warning alert.

At block 824, the user's level of engagement with the AWP 150 may be assessed or referenced. In examples where the user's engagement has increased, the user may receive a proactive alert (e.g., step 830). Conversely, in examples where the user's engagement level has not increased, the user may receive a warning alert to help improve wellness of the user (e.g., step 832).

Now referring back to step 814, of the previous example, where the first user and the second user indicated on Monday that their mood was sad, the first user had three days with positive moods in a row, the first user may have also indicated that their energy level was ten corresponding to a maximum energy level, via process 600 of FIG. 6. Thus, the process 800 may proceed to block 824, where the user may receive a proactive alert, via display of the user device (e.g., device 111). Conversely, if the first user had indicated that their energy level was 0 corresponding to a low energy level at step 814, the process 800 may proceed to block 826, where the first user may receive a warning alert.

Although, the present description and figures may illustrate specific values of mood or energy to initiate or continue the processes of FIG. 7 and FIG. 8, it may be contemplated that any value suitable determined via group 130 or AWP 150 may be suitable for the processes. It may be appreciated that the alert processes described in FIG. 7 and FIG. 8 (e.g., alert process 700 and alert process 800 respectively) providing an alert to the user may utilize any of a variety of inputs such as engagement level, mood, energy level, or any other variable associated with wellness, and may be customizable as desired. The processes of FIG. 5, FIG. 6, may be performed concurrently, in tandem, simultaneously, stepwise, or in any combination thereof to the processes of FIG. 7 and FIG. 8.

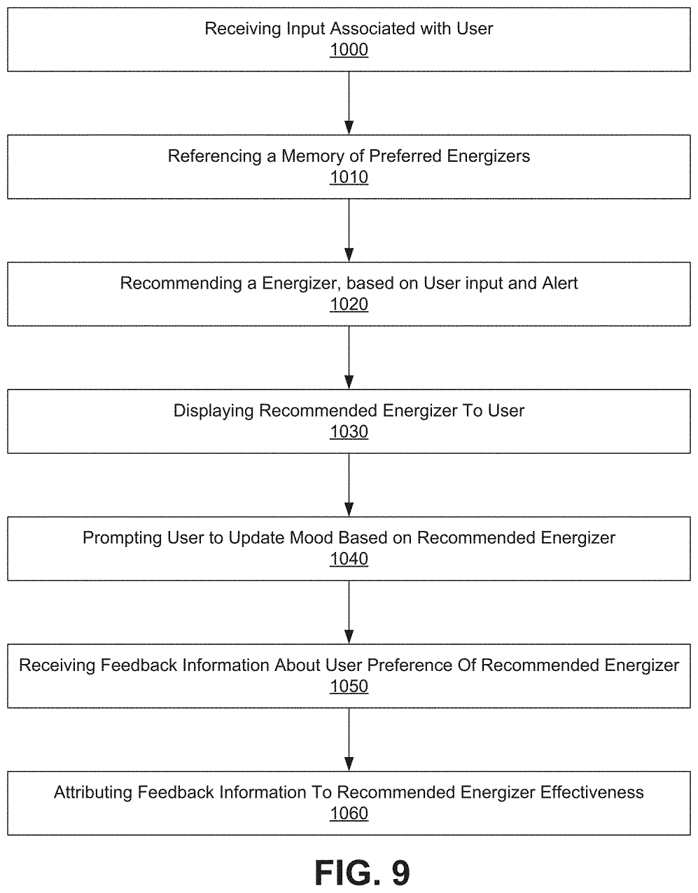

FIG. 9 illustrates an example method of initial energizer recommendation in accordance with an example of the present disclosure. The method of initial energizer recommendation may comprise receiving input associated with a user, wherein the input may be an energizer profile, mood information, energy level information, work environment, or any suitable wellness characteristic or any combination thereof; referencing a memory of preferred energizers; recommending an energizer, based on user input (e.g., work environment, energizer profile, mood, energy level, or a combination thereof and alert received via device 111; displaying recommended energizer to a user via display of device 111 associated with the user; prompting user to update mood and energy level based on performance of recommended energizer; receiving feedback information about user preference of recommended energizer; and attributing feedback information to recommended energizer effectiveness. Process 1000 is further described herein.

At step 1000, AWP 150 may receive input associated with a user such as, energizer profile, mood information, energy level information, environment (e.g., user location), or any suitable wellness attribute or some combination thereof, captured via device 111. At block 1010, the device 111 may reference a memory (e.g., non-removable memory 44 or removable memory 46) of preferred energizers associated with the energizer profile of the user or one or more users with similar profiles. In some alternative examples, the preferred energizers may be stored in a database (e.g., data store 165, 166) of the adaptive wellness system 160.

At block 1020, device 111 which may be communicatively linked to AWP 150 may recommend an energizer based on user input (e.g., combination of work environment, energizer profile, mood). Whereas the adaptive wellness platform may narrow down a list of energizers based on the preferred energizers associated with the user.

For example, a user's energizer profile may suggest that they prefer yoga, tic-tac-toe, relaxing outside as their preferred energizers, the user may have indicated that their mood is positive (e.g., connected, valued, happy, relaxed, etc.), the user may also have indicated their energy level to be five corresponding to average energy, and the user may be in an environment outside of work meaning they may have more time available to perform an energizer. The AWP 150 may determine based off the inputs described above that user may want to perform an energizer. In response, AWP 150 may communicate with a device (e.g., device 111) a list of energizers, where based on the energizer profile associated to the user may reduce the number of energizers in the list of energizers to a list of energizers that may be similar to a user's preferred energizers. The reduced list of energizers (may also be referred to as energizer recommendation) may be further reduced based on the mood, environment, energy level, or any combination thereof. In some examples, the environment may be determined via user input or selection via user interface of device 111. In alternate examples, environment (e.g., user location) may be determined via GPS chipset (e.g., GPS chipset 50) of the device 111.

At block 1030, device 111 may display one or more recommended energizers to the user. The recommended energizer may be displayed via image, video, alert, text, or any suitable method to display recommended energizer or any combination thereof. At block 1040, after a set amount of time or an indication of completed energizer by the user, device 111 may prompt the user to update mood or energy level based on the recommended energizer. Using the previous example, if the user is determined to be at home, the AWP 150 may have recommended relaxing outside as the recommended energizer. Following completion of the energizer, which may be indicated by the user via user interface of device 111, the user may be prompted to update mood level or energy level. The user may now indicate that their mood is connected, and their energy level is ten. In some examples, the user may also be prompted to provide their preference on the energizer they just performed or on other similar energizers to further define or add some variability to the user's energizer profile.

At block 1050, AWP 150 or device 111 may receive feedback information based on user preference of recommended energizer, where the feedback information may refer to the answers to a prompt displayed to the user at block 1050. The prompt may ask users to provide their preference on the recommended energizer performed or energizers similar to recommended energizers. Feedback information may also refer to the change in mood or energy level after the recommended energizer has been performed. In some alternate examples, feedback information may further comprise feedback information from a community (e.g., group 140), where AWP 150 may prompt other users, that the user may have indicated or selected as trusted members that constitute a group (e.g., group 140) to state their perception of change in the user's mood or energy level. Feedback information from a trusted member may be given a higher weight or priority in determinations of the user's mood, energy level, or the like. Trusted members are contemplated to generally be a subset of members of the one or more groups a user is a member of. The determination of a trusted member may be based on a selection by a user that indicates a member as trusted or other adaptive wellness platform information, such as likes, views, posts, comments, or other user actions or user roles.

At block 1060, AWP 150 may attribute feedback information to recommended energizer effectiveness, for example, if a certain energizer is shown to increase energy level by five, the five-point increase may be the assumed outcome of that energizer the next time a user performs that energizer, this concept may also refer to the change in mood.

For example, a user had an energy level of five prior to doing a recommended energizer (e.g., jogging, stretching, etc.). Once the user performed the recommended energizer, the user indicated that their energy level was now eight, but their mood did not improve. The recommended energizer that the user performed (e.g., jogging, stretching, etc.) may now be attributed to increasing energy level by three points but not changing the mood of the user, herein this attribution associated with the energizer may be referred to energizer effectiveness. So, in instances where a user may be three energy level points away from a preferred (e.g., threshold) energy level set by a company (e.g., group 130) the user may be prompted to perform the energizer that was attributed to a three-point increase in energy level.

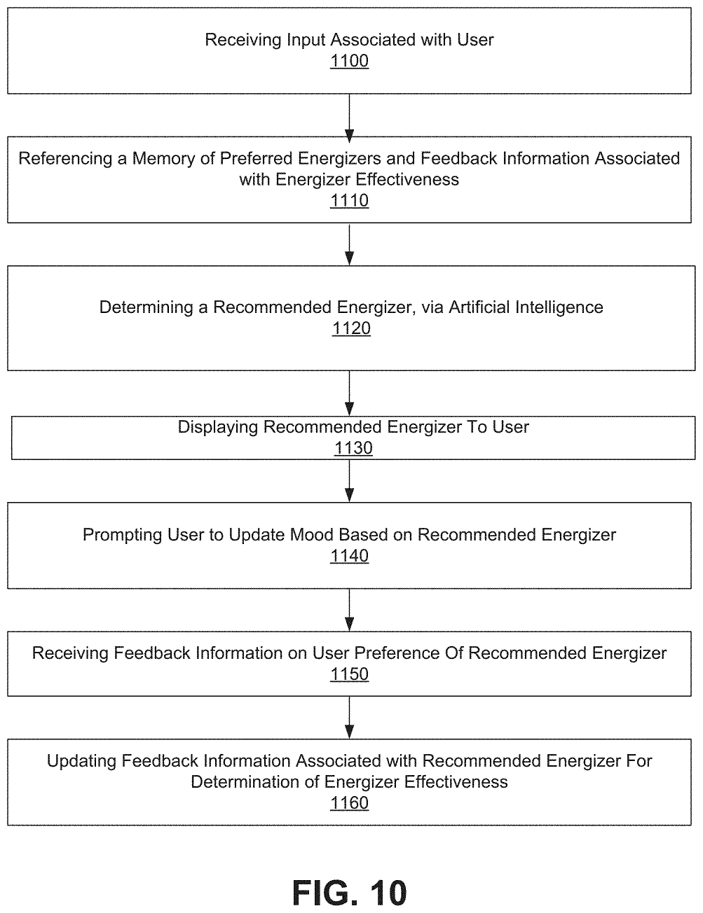

FIG. 10 illustrates an example method of recommending energizers. The method of recommending energizers may comprise receiving input associated with a user, wherein the input may be an energizer profile, mood information, energy level information, work environment, or any suitable wellness characteristic or any combination thereof; referencing a memory of preferred energizers and feedback information associated with energizer effectiveness; determining a recommended energizer, via artificial intelligence (e.g., machine learning), based on user input (e.g., work environment, energizer profile, mood, energy level, or a combination thereof alert received via device 111, and feedback information; displaying recommended energizer to a user via display of device 111 associated with the user; prompting user to update mood and energy level based on performance of recommended energizer; receiving feedback information about user preference of recommended energizer; and updating feedback information associated with recommended energizer performed for determination of energizer effectiveness. Process 1100 is further described herein.

At block 1100, AWP 150 may receive input associated with a user, such as, energizer profile, mood information, energy level information, work environment, or any suitable wellness attribute or some combination thereof, which may be captured via device 111. At block 1110, the device 111 may receive from a memory (e.g., non-removable memory 44 or removable memory 46) preferred energizers or feedback information associated with an energizer profile of the user. In some examples, the energizer profile may be stored in a database (e.g., data store 165, 166) of the adaptive wellness system 160.

At block 1120, device 111 communicatively linked to AWP 150 may determine a recommended energizer, via artificial intelligence (e.g., machine learning), based on input (e.g., combination of environment, energizer profile, mood, or other wellness information), alert, or feedback information received by the user. Whereas the AWP 150 may narrow down a list of energizers based on the preferred energizers associated with the user. In some alternate examples, device 111 communicatively linked to AWP 150 may make a determination to display multiple recommended energizers in order to achieve one or more wellness settings or preferences determined or set by group 130.

In an example, an energizer profile associated with a user may indicate jogging, meditating, or reading as one or more preferred energizers, the mood of the user may be indicated as relaxed (a positive mood), the energy level of the user may be indicated to be five corresponding to average energy, or the environment of the user may be indicated as being consistent with a work environment, which may translate to the user having less time available to perform an energizer. The AWP 150 may determine based off the inputs (e.g., energizer profile, mood, energy level, work environment, etc.) that user may want to perform an energizer. In response, AWP 150 may communicate a list of energizers to device 111. Based on the energizer profile associated with the user, the list of energizers may be reduced. The reduced list of energizers may be similar to a user's preferred energizers (80% of the preferred energizers are in the reduced list). The reduced list of energizers or preferred energizers may be further shortened or limited based on the feedback information associated with energizer effectiveness to pinpoint or select a specific recommended energizer that may increase overall wellness (e.g., mood, energy level, engagement level, etc.) to a particular level (e.g., a threshold level) consistent with settings or requirements defined by a company (e.g., group 130) associated with the user. Thus, based on the referenced energizer profile, feedback information, and preferred energizers, the AWP 150 may determine a recommended energizer based on the user input and company (e.g., group 130) settings or requirements to influence an increase in user wellness.

In an example, a company (e.g., group 130) has indicated that they would like the user's energy level to be eight and their mood to be at a maximum (e.g., ten), which corresponds to a mood of connected in the current example. At a first period, there may be indications that the user energy level is five, the user is in a work environment, and the user's mood is relaxed. Based on the indications in the first period, AWP 150 may make a determination to recommend an energizer that has been shown to increase the user's energy level by three (to reach the preferred threshold of eight) and increase the user's feeling of connectedness (e.g., increase the user's mood to a threshold level indicating connectedness or some increment towards that threshold level). The recommended energizer may be determined via feedback information of previous recommended energizer processes and user input such as, work environment, energizer profile, mood, energy level, etc. If a previous recommended energizer process (e.g., process of FIG. 9 or FIG. 10) has shown that in the work environment listening to music increases the user's feeling of connectedness to maximum and energy level by three points, the adaptive wellness system may recommend that the user listen to music.

At block 1130, device 111 may display or otherwise communicate the recommended energizer. The recommended energizer may be displayed via image, video, alert, text, or any suitable method to display recommended energizer or any combination thereof. At block 1140, after a set amount of time or indication of completed energizer, device 111 may prompt the user to update mood and energy level based on the recommended energizer, via notification or alert on the display of a device 111 associated with the user.

In the example of the previous paragraph, when a recommended energizer has been determined it may be displayed via the display of a user device (e.g., device 111). Based on a period of time indicative to an energizer being performed, the user indicating that the energizer was performed, or another means of indication, device 111 may display message for the user to update their preference on the energizer that was just performed or similar energizers.

At block 1150, AWP 150 or device 111 may receive feedback information based on user preference of recommended energizer, where the feedback information may refer to the answers to the prompt displayed to the user via user interface at block 1150. The prompt may ask users to provide their preference on the recommended energizer performed or energizers similar to recommended energizers. Feedback information may also refer to the change in mood or energy level after the recommended energizer has been performed. In some alternate examples, feedback information may further comprise feedback information from a group (e.g., group 140), where AWP 150, may prompt other users, that the user may have indicated or selected as trusted members that constitute a group (e.g., group 140) to state their perception of change in the user's mood or energy level.

At block 1160, AWP 150 may update feedback information associated with the recommended energizer for determination and update to energizer effectiveness, for example, if a certain energizer is shown to increase energy level by two, the two-point increase may be the assumed outcome of that energizer the next time a user performs that energizer, this concept may also refer to the change in mood.

For example, a user had an energy level of three prior to doing a recommended energizer (e.g., jogging, stretching, etc.). Once the user performed the recommended energizer, for example jogging, the user indicated that their energy level was now 7 but their mood did not improve. The recommended energizer that the user performed (e.g., jogging) may now be attributed to increasing energy level by four points but not changing the mood of the user. So, in instances where a user may be four energy level points away from a preferred energy level set by a company (e.g., group 130) the user may be prompted to perform the energizer that was attributed to a four-point increase in energy level.

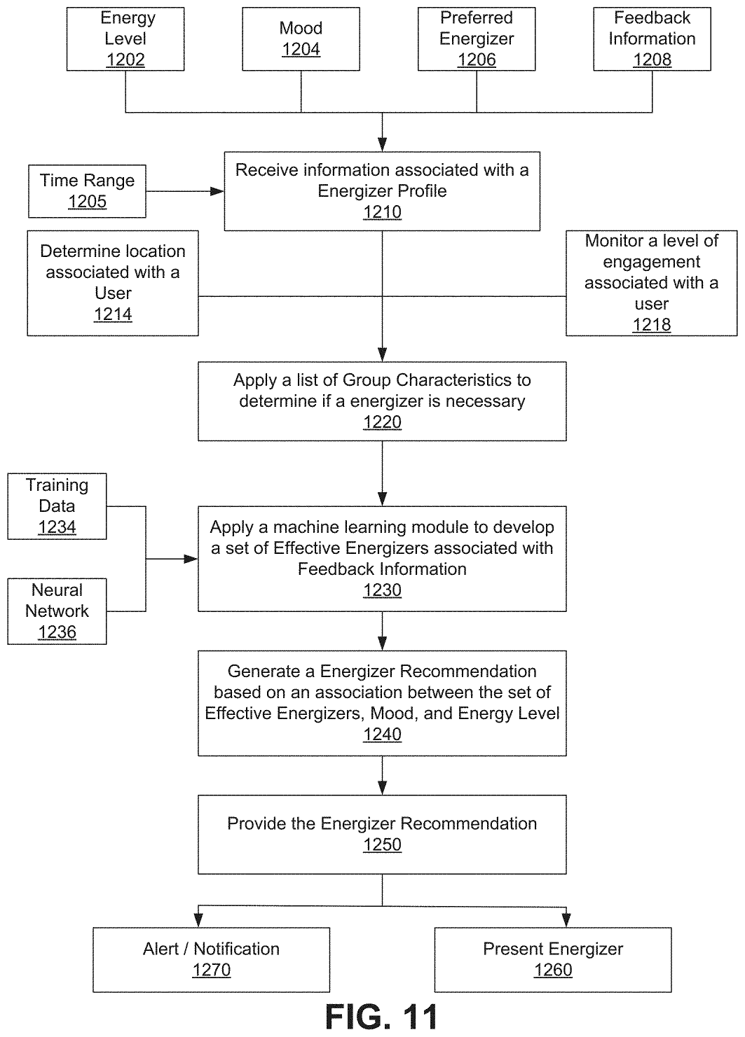

FIG. 11 illustrates a flow chart for monitoring wellness associated with a user in accordance with an example of the present disclosure. At block 1210, a device (e.g., device 111 of FIG. 1) may receive information associated with an energizer profile. The energizer profile may be associated with a particular user, wherein the energizer profile may comprise user characteristics such as, energy level 1202, mood 1204, preferred energizers 1206, feedback information 1208, user location 1214, level of engagement 1218, or the like.

At block 1205, a device (e.g., device 111) may facilitate a time range of user characteristics, such as energy level 1202, mood 1204, preferred energizers 1206, feedback information 1208, user location 1214, level of engagement 1218, or the like, to be received or incorporated into an energizer profile. In some examples, energy level 1202 or preferred energizers 1206 may be determined via an energy level capture process (e.g., process 500 of FIG. 5). In some other examples, mood 1204 may be determined via a mood capture process (e.g., process 500 of FIG. 5). In another example, feedback information may be determined via an energizer recommendation method as shown in FIG. 9 or FIG. 10. In yet another example, user location 1214, may be indicated by a user via user interface. In some examples, user location 1214 may be determined via GPS chipset (e.g., GPS chipset 50 of FIG. 2) of a device (e.g., device 111 of FIG. 2). In various examples, the level of engagement 1218 (which may be presented as a numerical value) may be determined by content posted (e.g., tips to co-workers, asks, or shares), published, or otherwise interacted with, within a particular date range may be extracted from a user profile or energizer profile. Interactions may include, but are not limited to a like, view, post, comment, or other user action with respect to the content. The time range may be associated with a time stamp associated with a particular piece of input (e.g., energy level 1202, mood 1204, preferred energizer 1206, feedback information 1208, user location 1214, level of engagement 1218, or etc.). As such, the content time range may relate to a date or time a portion of the input was updated, created, posted, published, or interacted with. In some examples, the time range may indicate a most recent energy level update, mood update, post, publication, or other user interaction with an input within a number of hours, days, or months. For example, a time range of received inputs may correspond to information posted by a user on the AWP 150 within the past six months. In another example, the time range may be a three-month window corresponding to a season, such as summer. As such, inputs associated with the energizer profile during that three-month window may correspond to the user's summer activity.

Input time ranges may be defined manually, automatically, or dynamically, to enable customization. Since users' adaptive wellness activity, lifestyle, interests, or overall wellness may change over time (e.g., months or years) and user's may be more active at certain times or user's in certain roles within a company (e.g., group 130) may have more time to interact with the adaptive wellness system, specifying a time range for received inputs may assist in greater accuracy and current relevance of wellness while users are conducting work associated with a company (e.g., group 130 of FIG. 2).

For example, it may be desirable, e.g., to a group, to identify relevant time ranges associated with users' activity, and specifically target capturing wellness during particular hours important to users or a group of users (e.g., group 130). Specifying a time range to analyze inputs within a most recent number of days, months, or years may assist in identifying users who actively enjoy or participate group (e.g., group 130 or group 140) driven efforts.

At block 1220, a device (e.g., device 111) or AWP 150 may apply a list of group characteristics, associated with 5R, to determine if an energizer should be used to improve wellness of a user, employees, community, etc. A profile of a group 130 (e.g., a company) may include what wellness levels the group would prefer represented with their users (e.g., employees). The list of group characteristics may include a ranked set of behavioral categories associated with 5R that the company (e.g., group 130) finds relevant values or goals.

In various examples, the list of group characteristics may rank or prioritize certain behavioral categorizes from the inputs, based on one or more categories such as roles, respect, rules, recognition, routines, or the like. A behavioral category may be repeatedly identified in the received inputs, which may indicate a greater interest or interaction between users (e.g., employees), and AWP 150. The level of interaction with a particular category may be associated with a particular input, and further assist in various operations discussed herein.

At block 1230, a machine learning module (e.g., machine learning model 1410) may be applied to determine a set of effective energizers associated with feedback information or preferred energizers. In some examples, a device (e.g., device 111) may execute the machine learning module (e.g., associated with block 1230). One or more machine learning modules may analyze energizer profile(s) to determine effective energizers relevant or the users' wellness. In various examples, the machine learning module may utilize training data 1234 to develop and predict associations between an energizer profile and an effective energizer. The training data 1234 may be previously analyzed or associated with feedback information or effective energizers. In other examples, the training data may be associations between energizer profiles, feedback information, or indicated effective energizer.

A neural network 1236 may assist in utilizing the training data 1234 or assisting with the machine learning techniques to develop the set of effective energizers associated with the feedback information. For example, the neural network may be trained on data indicating various types of energizer profiles and feedback information (see, e.g., FIG. 5A-5C, 6A-6C, 9, or 10), and a set of effective energizers associated with each energizer profile and its respective feedback information. When an energizer profile is identified via the received inputs (e.g., energy level 1202, mood 1204, preferred energizers 1206, level of engagement 1218, or feedback information 1208), a set of one or more effective energizers may be associated with the energizer profile. In an example, an extracted energizer profile and its respective feedback information. The machine learning module may associate a positive change in mood, energy level, or level of engagement, with energizers but not limited to, the specific performed energizer that may have been determined to be effective.

At block 1240, a device (e.g., device 111) may generate energizer recommendation(s) based on an association between the set of effective energizers, mood, or energy level. The energizer recommendation may utilize data directly from the machine learning module. In examples, the effective energizers may be compiled in an energizer recommendation(s) (e.g., alerts to device 111). The energizer recommendation(s) may include a list of energizers which may be based on preferred energizers and energizer effectiveness related to an increase in mood, an increase in energy level, or an increase in engagement level related to interactions with AWP 150. In various examples, the set of one or more effective energizers related to the energizer profile associated with a user and the list of group characteristics may develop the generation of the recommended energizer. In some examples, where multiple inputs of the energizer profile and corresponding set of effective energizers are identified, the energizer recommendation may be representative of an effective energizer that may increase the majority of inputs or one or more effective energizers that increase each input. Since different group characteristics may indicate increased importance of some inputs associated with the energizer profile, the energizer recommendation may be determined based on an indicated weight or priority of an increase of a particular input in respect to the list of group characteristics associated with behavioral categories (e.g., 5R). As such, the energizer recommendation may include recommendations for improving wellness of a user.

At block 1250, a device (e.g., device 111) may provide the energizer recommendation, for example, by an AWP 150, to a user's computing device or the like. The presentation of the energizer recommendation 1260 may be provided by a device (e.g., device 111, 131, 132, 133, 134, 141, 142, 143, 144) in the form of an alert, a notification, image, video, instructional text, email, message, among others. A alert/notification 370 may similarly be presented by a device (e.g., device 111, 131, 132, 133, 134, 141, 142, 143, 144) in a variety of forms to a user's computing device (e.g., such as text or banner on a home screen, pop-up alert, post on a user feed within the AWP 150, a message, email, haptic feedback an alert or notification within an app) when interacting with a collaborative platform, or the like.

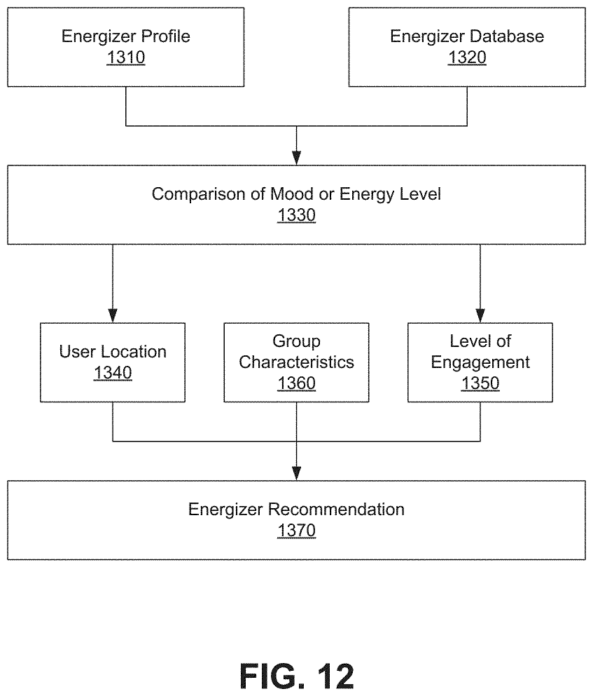

FIG. 12 illustrates a flow chart for generating an energizer recommendation, in accordance with an example of the present disclosure. At block 1310, a device (e.g., device 111) may generate an energizer profile with the various techniques and processes described herein. At block 1320, an energizer database may provide a compilation of energizers available for presentation to a user, e.g., a list of actions that may affect wellness. In some examples, the energizer database may be embodied within a device (e.g., device 111). In some other examples, the energizer database may be formulated externally from the device via server (e.g., server 161, 162) or stored via data store (e.g., data store 165, 166) of the adaptive wellness system 160.

At block 1330, a device (e.g., device 111) may perform a mood or energy level comparison to a preferred mood or energy level set by a company (e.g., group 130) associated with the user and may compare energizer database to a user location associated with the device (e.g., device 111, 131, 132, 133, 134, 141, 142, 143, 144) to truncate or preface energizer database further based on user location at block 1340. In some examples, the user location may determine the time a user may have available to perform an energizer. At block 1350, a device (e.g., device 111) may reference monitored interactions with adaptive wellness system, where an interaction such as a like, view, post, comment, or other user action with respect to the content on the server of the adaptive wellness system 160 may be monitored. In some examples, at block 1360 a device (e.g., device 111, 131, 132, 133, 134, 141, 142, 143, 144) may combine a list of group characteristics with the user location determination and level of engagement to develop one or more energizer recommendation(s) at block 1370.

In some examples, a device (e.g., device 111) may develop one or more energizer recommendation(s) at block 1370. The list of group characteristics may include one or more rules or weights to behavioral categories associated with the user and company (e.g., group 130) values or goals. For example, some inputs of the energizer profile may be weighted greater for a group A, despite having a lower relevance for group B. Group A may value a high sense of connectedness within their employees (e.g., users), whereas group B may value a higher energy level in comparison to group A. For example, a user works with both group A and group B, two different energizers may be recommended for the user because there may be two different energizer databases with corresponding minimum or maximum thresholds for energizer related information or other group characteristics. The analyzed energizer profiles for group A and group B may be identical with identical respective inputs that indicate a higher relevance for energizer A with group A, and energizer B with group B. Group B may not recommend Group A's energizer because the values and goals determined by Group B relate to a higher energy level whereas Group A relates to a higher mood. In this instance Group B's weight regarding energy level may be higher than other inputs. Whereas for Group A, the weight may higher for connectedness associated with a user therefore other inputs may not be worth as much in the adaptive wellness system associated with Group A.

Energizer recommendation(s) may be provided through various mechanisms and methods, as discussed herein. Energizer recommendations may be determined using any of a variety of techniques and considerations. As such, there exists flexibility and customizability in determining recommended energizers and applicability to various users or groups (e.g., communities or companies).

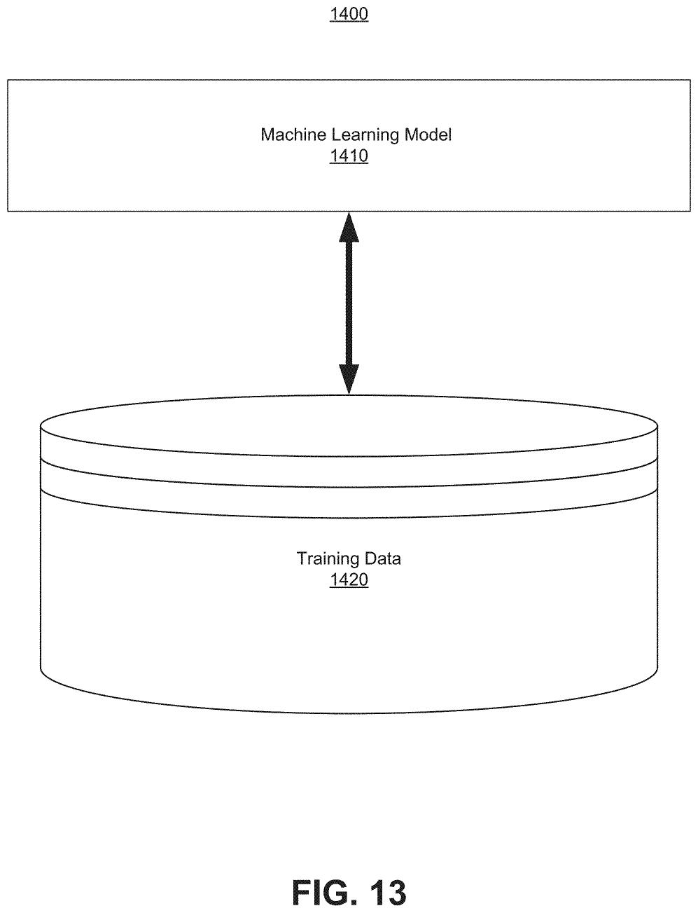

FIG. 13 illustrates a framework 1400 that may be employed by AWP 150 associated with machine learning. The framework 1400 may be hosted remotely. Alternatively, the framework 1400 may reside within the adaptive wellness system 160 as shown in FIG. 1, or be processed by the device 111. The machine learning model 1410 may be operably coupled with the stored training data in a database (e.g., data store 163). In some examples, the machine learning model 1410 may be associated with operations of FIGS. 5A-12. In some other examples, the machine learning model 1410 may be associated with other operations. The machine learning model 1410 may be implemented by one or more machine learning module(s) or another device (e.g., server 162 or device 111).

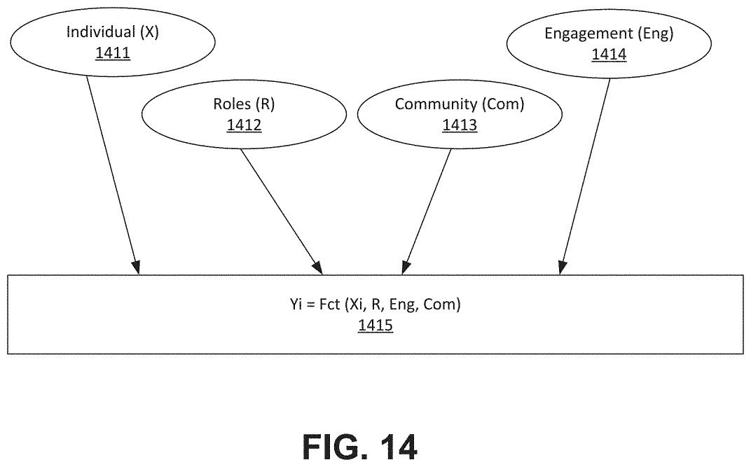

There may be many different categories of wellness information that may serve as data input. As shown in FIG. 14, the categories of wellness information may include individual (X) 1411, roles (R) 1412, engagement (Eng) 1413, or community (Com) 1414. Individual category (X) may include class profile, energizer profile, or diversity (e.g., the practice or quality of including or involving people from a range of different social and ethnic backgrounds and of different genders, etc.). The role category (R) may include a team role, a collaboration role, or a community member role. The engagement category (Eng.) may indicate activities in numbers or ratios and may include tips, asks, shares, comments, or people first contribution. Community category (Com) may include a project theme, type, or diversity index. Data output (Y) may include an indication of mood, energy, or the like. Information such as the individual or the period (e.g., day or week average) may be consider or noted for the data output.

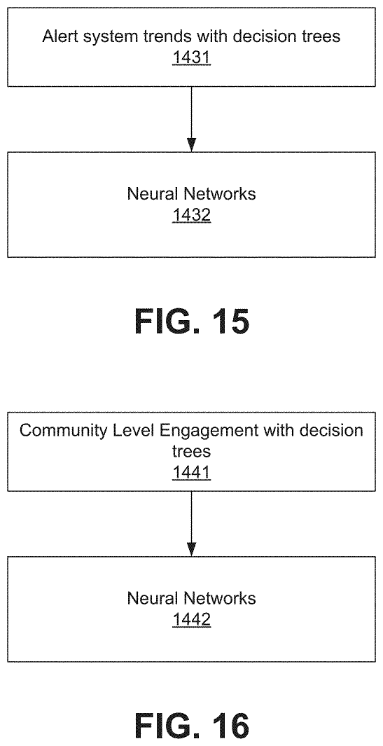

The data input or data output may be associated with different functions, which may be on an individual level, associated with an alert system, or community level. At an individual level the function 1415 may be Yᵢ=fct(Xᵢ, R, Eng, Com). An alert system of AWP 150 may determine trends of function Y using machine learning. As shown in FIG. 15, for example, at step 1431 operations (e.g., algorithms or modules) with decision trees, such as in FIG. 7 or FIG. 8, may be implemented. Such operation thresholds or sequences may evolve using machine learning techniques. At step 1432, a neural network machine learning model may be used to make decisions associated with the alerts that may be sent, which may be based on the trends corresponding to the decision tree operation.

A community level function of AWP 150 may be represented as Eng=Fct (Com, 5R, average Yᵢ). This engagement function may help interpret or react to the engagement level for a group (e.g., group 140) or an individual (e.g., user associated with device 111). As shown in FIG. 16, for example, at step 1441 operations with decision trees, such as in FIG. 7 or FIG. 8, may be implemented. Such operation thresholds or sequences may evolve using machine learning techniques. At step 1442, a neural network machine learning model may be used to make decisions associated with determining engagement level (e.g., engagement function may evolve), which may be based on the trends corresponding to the decision tree operation.

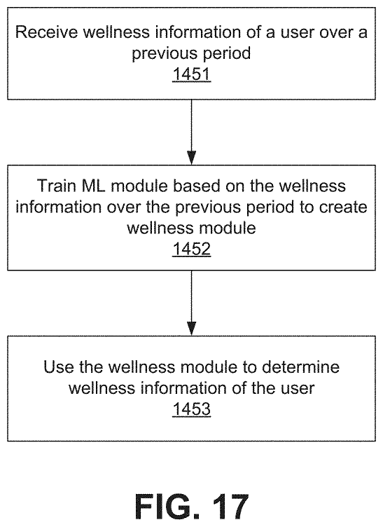

FIG. 17 illustrates an exemplary method for using machine learning to determine wellness information. At step 1451, device 111 may receive wellness information associated with a user of device 111. The user may be associated with a user profile linked to group 130 of AWP 150. As disclosed in herein AWP may be defined by a 5R behavioral model. At step 1452, training a machine learning module on the wellness information associated with the user at a plurality of previous periods in order to determine a wellness module (e.g., algorithm or operation). The wellness module may help determine (e.g., current or predicted) wellness information of the user of device 111 or group 130 during one or more subsequent periods. The wellness module may continually evolve. At step 1453, the wellness module may determine subsequent wellness information of the user at some time t1. As disclosed herein, wellness information may include mood associated with the user, energy level associated with the user, or a level of engagement of the user. In an example, wellness module may help predict the energy level of a user for the remaining day, remaining week, or month if an energizer is used or not used. In another example, the wellness module may be used to help predict how an energizer activity may affect a user's mood. Therefore, particular energizers may be sent in an alert message to device 111 based on a threshold mood that is preferred for the user.

Additional examples regarding the use of machine learning with AWP 150, is disclosed herein. In an example, the training data 1420 may include attributes of thousands of objects. For example, the objects may include a smart device (e.g., device 111), person, book, newspaper, sign, car, content items (e.g., posts, messages, notifications, images, videos, audio), or the like. Attributes may include but are not limited to the size, shape, orientation, position of the object, etc. The training data 1420 employed by the machine learning model 1410 may be fixed or updated periodically. Alternatively, the training data 1420 may be updated in real-time based upon the evaluations performed by the machine learning model 1410 in a non-training mode. This is illustrated by the double-sided arrow connecting the machine learning model 1410 and stored training data 1420.

In some examples, the machine learning model 1410 may be trained to determine one or more forward looking projections of a forecasted or predicted energizer profile associated with one or more users of a network based in part on analyzing one or more historical signals or data items (e.g., objects) associated with the users. Signals or data items may refer to data or items associated with the components of one or more energizer profiles, such as mood, energy level, level of engagement, user location, or the like, which may be wellness information. In some examples, the historical signals or data items may be associated with one or more accuracy levels, one or more integrity values, or the like in the manner described above. In this regard, the machine learning module 1410 may generate short-term predictions (e.g., a couple days or weeks) of energizer recommendations or long-term predictions (e.g., a couple months or more) of energizer recommendations associated with one or more users of AWP 150.

In addition to the benefits described herein, the examples of the present disclosure may facilitate extraction of an energizer profile from a user or group's adaptive wellness platform (e.g., AWP 150). A main challenge in doing so is determining a manner in which to ensure that an extracted representation is indeed relevant to an effective energizer associated with the user, group, or company.

Aspects of the present disclosure, including the energizer recommendations, may deploy a machine learning model that is flexible, adaptive, automated, learns quickly, or trainable. The disclosed subject matter may incorporate self-supervised learning framework or set-embedding neural network model aspects. As such, this may enable the AWP 150, which may include effective energizers of the examples of the present disclosure, to be flexible and scalable to billions of users, and their associated devices on the AWP 150.

Moreover, examples of the present disclosure may utilize automated machine learning models, in part, to achieve the goal of analyzing disparate forms, types, or features of, or within, online content, such as user generated content, or extracting patterns, styles, characteristics, or an overall energizer profile. Energizer profile may provide a common format which may be compared, contrasted, or analyzed by automated machine learning models of the present disclosure, thus enabling diverse types of online content to be analyzed. Such techniques may significantly improve efficiencies with respect to review, processing, or analysis, reducing or eliminating a need for manual review or subjective determinations, or provide techniques for automation.

In example, the automated machine learning models may be trained, based on input training data associated with identifying of different types of user characteristics (e.g., mood, energy level, engagement level, etc.), and the automated machine learning models may be provided inputs of different weights and values via a list of group characteristics, to enable customization for certain features and aspects which may be of interest. In this regard, the automated machine learning models may be configured to (automatically) analyze, extract, or identify content items or features in an efficient, streamlined manner, or generate a common format (e.g., energizer profile to enable comparisons between disparate features, inputs, or aspects). As such, individuals and devices need not analyze content in a computationally intensive, e.g., brute force manner, as may exist in some conventional systems, which inefficiently drains or constrains processing resources or capacity of devices. On the other hand, the automated machine learning models of the present disclosure being trained based on the input training data described herein to provide tailored energizer recommendations, which may enable reduction/conservation of processing capacity of devices by being able to determine the energizer recommendation in an automated and dynamic manner. The conservation of the processing capacity of the devices by implementing the automated machine learning models of the present disclosure may improve the operation and functionality of the devices of the present disclosure. Additionally, based on the automated machine learning models' ability to quickly learn and predict features, items, styles, etc., over time, the accuracy of the predictions of the energizers or group characteristics may be significantly increased and thereby significantly improve or assess a user's characteristics (e.g., mood).

The disclosed subject matter may have a technical effect of periodically or continually evolving the collaborative platform (e.g., AWP 150) for an individual user or a group. This may lead to automated updates to the collaborative platform that may be tailored over time to each group or user associated with the collaborative platform. The disclosed subject matter may allow for timely alerts that encourage and increase collaboration among a group of users.

It is to be appreciated that examples of the methods and apparatuses described herein are not limited in application to the details of construction and the arrangement of components set forth in the following description or illustrated in the accompanying drawings. The methods and apparatuses are capable of implementation in other examples and of being practiced or of being carried out in various ways. Examples of specific implementations are provided herein for illustrative purposes only and are not intended to be limiting. In particular, acts, elements and features described in connection with any one or more examples are not intended to be excluded from a similar role in any other examples. It is contemplated that methods may apply to the user or to the group. For example, energizer related alerts may be determined by the groups mood or other wellness information. Energizers may be group related activity rather than an individual related activity.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The foregoing description of the examples has been presented for the purpose of illustration; it is not intended to be exhaustive or to limit the patent rights to the precise forms disclosed. Persons skilled in the relevant art can appreciate that many modifications and variations are possible in light of the disclosure.

Some portions of this description describe the examples in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one example, a software module is implemented with a computer program product comprising a computer-readable medium containing computer program code, which can be executed by a computer processor for performing any or all of the steps, operations, or processes described.

The scope of this disclosure encompasses all changes, substitutions, variations, alterations, and modifications to the example examples described or illustrated herein that a person having ordinary skill in the art would comprehend. The scope of this disclosure is not limited to the example examples described or illustrated herein. Moreover, although this disclosure describes and illustrates respective examples herein as including particular components, elements, feature, functions, operations, or steps, any of these examples may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular examples as providing particular advantages, particular examples may provide none, some, or all of these advantages.

Examples also may relate to an apparatus for performing the operations herein. This apparatus may be specially constructed for the required purposes, or it may comprise a computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Examples also may relate to a product that is produced by a computing process described herein. Such a product may comprise information resulting from a computing process, where the information is stored on a non-transitory, tangible computer readable storage medium and may include any example of a computer program product or other data combination described herein.

Finally, the language used in the specification has been principally selected for readability and instructional purposes, and it may not have been selected to delineate or circumscribe the inventive subject matter. It is therefore intended that the scope of the patent rights be limited not by this detailed description, but rather by any claims that issue on an application based hereon. Accordingly, the disclosure of the examples is intended to be illustrative, but not limiting, of the scope of the patent rights, which is set forth in the following claims.

According to various aspects of the present disclosure, systems and methods may utilize artificial intelligence (AI) to generate a set of effective energizers, also referred to herein as a “effective energizer.” The effective energizer may broadly refer to an energizer that may be attributed to an update in mood, energy level, level of engagement, or any component of the energizer profile or any combination thereof. For example, jogging may be considered an effective energizer if it has been attributed to an update in mood, energy level, level of engagement, or one or more component of an energizer profile. Implementations of the present disclosure may identify energizers similar to a user's preferred energizers, as well as updates to inputs or components of an energizer profile(s) and use such information to generate a set of effective energizers to match the situation or wanted increase in energizer profile inputs (e.g., mood, energy level, level of engagement, work environment, etc.). Accordingly, various implementations may infer and associate an effective energizer shown to improve energizer profile for a user, then match that effective energizer to the effective energizer of a similar user.

In one example, systems or methods may infer that if a user's energizer profile updates positively when the energizer is solving puzzles, it may be inferred that the user may like energizers that involve problem solving. In response, the AI operations may suggest a similar energizer such as solving puzzles, playing chess, or the like to another user who may also show a positive update to their energizer profile when energizer involves problem solving. AI operations, as discussed herein may determine such an association between an effective energizer associated with a user and match that with effective energizers for another similar user.

Methods, systems, and apparatuses, among other things, as described herein may provide for adaptive wellness platform. A method, system, computer readable storage medium, or apparatus provides for receiving information associated with an energizer profile associated with a user on an adaptive wellness platform; applying a list of characteristics to determine when to suggest an energizer; applying a machine learning module to develop a set of effective energizers associated with the energizer profile and a feedback information associated with at least one energizer; generating an energizer recommendation based on an association between the set of effective energizers and the energizer profile; and providing the energizer recommendation via the adaptive wellness platform. The method, system, computer readable storage medium, or apparatus provides for training the machine learning module based on a set of energizers associated with at least one behavioral characteristic of the list of characteristics, wherein the list of characteristics is defined by the adaptive wellness platform, or a group associated with the user. The machine learning module may use a neural network (which may be used for clustering) to develop an association for the energizer profile, the list of characteristics, alert system trends, engagement (e.g., community level or individual), or one or more effective energizers, among other things. The energizer profile may include at least one of a mood, an energy level, a level of engagement, a preferred energizer, the feedback information, or a location associated with the user. A first effective energizer in the set of effective energizers may relate to at least one of an updated energizer profile or a preferred energizer. The received information (e.g., wellness information) may include at least one a mood, an energy level, a level of engagement, or a preferred energizer, the feedback information, or a location associated with the user. The energizer recommendation may be an alert, such as a notification, image, video, text, email, or message, to facilitate performing the energizer. The method, system, computer readable storage medium, or apparatus provides for displaying the energizer recommendation as an alert, a notification, image, or video during an interaction on the adaptive wellness platform. The method, system, computer readable storage medium, or apparatus provides for the energizer recommendation on a graphical user interface of a device. The received information may include information associated with the energizer profile within a time range. The preferred energizer may be associated with a preference based on the user. All combinations in this paragraph and the previous paragraphs (including the removal or addition of steps) are contemplated in a manner that is consistent with the other portions of the detailed description.

Methods, systems, and apparatuses, among other things, as described herein may provide for adaptive wellness platform. A method, system, computer readable storage medium, or apparatus provides for receiving, by a device, wellness information associated with a user, wherein the user is associated with a user profile linked to a first group of a plurality of groups of a collaborative platform (e.g., social media platform); based on the indication of the wellness information associated with the user, sending, by the device, an alert; receiving, by the device, an indication of a selection of an activity associated with the alert, wherein the activity comprises an energizer activity; receiving, by the device, feedback information associated with the activity, wherein the feedback information is linked to the user profile; and transmitting, by the device, a second alert based on the feedback information. The transmitting of the second alert may be based on training a machine learning module on a plurality of feedback information, wherein the plurality of feedback information comprises the feedback information associated with the activity. The machine learning module may utilize a neural network or small data operations (e.g., algorithms) at the beginning (depending on best results and number of data) to develop an association between a user profile and the activity. The collaborative platform may be defined by a 5R behavioral model for a group of user profiles. The wellness information comprises a mood associated with the user or an energy level associated with the user, a level of engagement, a preferred energizer, the feedback information, and a location associated with the user. The wellness information comprises a level of engagement associated with the user or a preferred energizer associated with the user. The wellness information may include a calculable value illustrated by a vector, such as a mood value. The feedback information may include feedback information from a trusted member. The trusted member feedback information is weighted differently compared to other feedback information. The trusted member is determined based on user-indicated selection of a member, likes, views, posts, or comments associated with the first group the collaborative platform. All combinations in this paragraph and the previous paragraphs (including the removal or addition of steps) are contemplated in a manner that is consistent with the other portions of the detailed description.

A method, system, or apparatus may provide for receiving, by a device, wellness information associated with a user, wherein the user is associated with a user profile linked to a first group of a collaborative platform, wherein the collaborative platform is defined by a 5R behavioral model; and training a machine learning module on the wellness information associated with the user at a plurality of previous periods in order to determine a predictive wellness module, wherein the predictive wellness module helps predict wellness information during a subsequent period. A method, system, or apparatus may provide for receiving, by a device, wellness information associated with a user profile, wherein the user profile is linked to a group of a collaborative platform, wherein the collaborative platform is defined by a 5R behavioral model; training a machine learning module on the wellness information associated with the user at a plurality of previous periods in order to determine a wellness module, wherein the wellness module helps determine wellness information of the user or the group during a subsequent period; and using the wellness module to determine subsequent wellness information of the user. Based on the subsequent wellness information, an alert may be sent. The wellness module may be an updated version of the machine learning module. The alert may include an activity for the user to complete. All combinations in this paragraph and the previous paragraphs (including the removal or addition of steps) are contemplated in a manner that is consistent with the other portions of the detailed description.

What is claimed:

1. A method comprising:
receiving, by a device, wellness information associated with a user, wherein the user is associated with a user profile linked to a first group of a plurality of groups of a collaborative platform;
based on an indication of the wellness information associated with the user, sending, by the device, an alert;
receiving, by the device, an indication of a selection of an activity associated with the alert, wherein the activity comprises an energizer activity;
receiving, by the device, feedback information associated with the activity, wherein the feedback information is linked to the user profile; and
transmitting, by the device, a second alert based on the feedback information.

2. The method of claim 1, wherein the transmitting of the second alert is based on training a machine learning module on a plurality of feedback information, wherein the plurality of feedback information comprises the feedback information associated with the activity.

3. The method of claim 2, wherein the machine learning module utilizes a neural network to develop an association between the user profile and the activity.

4. The method of claim 1, wherein the wellness information comprises a level of engagement associated with the user or a preferred energizer associated with the user.

5. The method of claim 1, wherein the wellness information comprises a calculable value illustrated by a vector.

6. The method of claim 1, wherein the feedback information comprises feedback information from a trusted member, wherein the feedback information from the trusted member is weighted differently compared to other feedback information, wherein the trusted member is determined based on user-indicated selection of a member, likes, views, posts, or comments associated with the first group of the collaborative platform.

7. The method of claim 1, wherein the collaborative platform is defined by a 5R behavioral model.

8. A method comprising:
receiving, by a device, wellness information associated with a user profile, wherein the user profile is linked to a group of a collaborative platform, wherein the collaborative platform is defined by a 5R behavioral model;
training a machine learning module on the wellness information associated with the user profile at a plurality of previous periods in order to determine a wellness module, wherein the wellness module helps determine wellness information of a user associated with the user profile or the group during a subsequent period; and
using the wellness module to determine subsequent wellness information associated with the user.

9. The method of claim 8, further comprising based on the subsequent wellness information, sending an alert, wherein the alert comprises an activity for the user to complete.

10. The method of claim 8, wherein the machine learning module utilizes a neural network or small data operations.

11. The method of claim 8, wherein the wellness information comprises a mood associated with the group or level of engagement associated with the group, wherein the group comprises a plurality of different user profiles.

12. The method of claim 8, wherein the wellness information comprises a level of engagement associated with the user or a preferred energizer associated with the user.

13. The method of claim 8, wherein the wellness information comprises a calculable value illustrated by a vector.

14. The method of claim 8, wherein the machine learning module is further trained using likes, views, posts, or comments associated with the group.

15. A system comprising:
one or more processors; and
one or more memory coupled with the one or more processors, the one or more memory storing executable instructions that when executed by the one or more processors cause the one or more processors to effectuate operations to:
receiving wellness information associated with a user profile, wherein the user profile is linked to a group of a collaborative platform, wherein the collaborative platform is defined by a 5R behavioral model;
training a machine learning module on the wellness information associated with the user profile at a plurality of previous periods in order to determine a wellness module, wherein the wellness module helps determine wellness information of a user associated with the user profile or the group during a subsequent period; and
using the wellness module to determine subsequent wellness information associated with the user.

16. The system of claim 15, further comprising based on the subsequent wellness information, sending an alert, wherein the alert comprises an activity for the user to complete.

17. The system of claim 15, wherein the machine learning module utilizes a neural network or small data algorithms.

18. The system of claim 15, wherein the wellness information comprises a mood associated with the group or level of engagement associated with the group, wherein the group comprises a plurality of different user profiles.

19. The system of claim 15, wherein the wellness information comprises a level of engagement associated with the user or a preferred energizer associated with the user.

20. The system of claim 15, wherein the wellness information comprises a calculable value illustrated by a vector.
